(19) United States
(12) Patent Application Publication
(10) Pub. No.: US 2010/0258440 A1
(43) Pub. Date: Oct. 14, 2010

(54) ANALYSIS APPARATUS AND ANALYSIS METHOD FOR CAPILLARY ELECTROPHORESIS

(75) Inventors: Koji Sugiyama, Kyoto (JP); Yukio Higashiisokawa, Kyoto (JP); Yusuke Nakayama, Kyoto (JP); Yoshihide Tanaka, Osaka (JP)

Correspondence Address: MORGAN LEWIS & BOCKIUS LLP, 1111 PENNSYLVANIA AVENUE NW, WASHINGTON, DC, 20004, US

(73) Assignees: ARKRAY, Inc., Kyoto (JP); NATIONAL INSTITUTE OF ADVANCED INDUSTRIAL SCIENCE AND TECHNOLOGY, Tokyo (JP)

(21) Appl. No.: 12/506,917

(22) Filed: Jul. 21, 2009

(30) Foreign Application Priority Data
Jul. 22, 2008 (JP) 2008-188840

(51) Int. Cl. C07K 1/26

(52) U.S. Cl. 204/451

(57) ABSTRACT

A capillary electrophoresis analysis apparatus is provided for analyzing samples by a capillary electrophoresis method that allows for rapid and highly accurate separation and detection, wherein the apparatus may be used in the diagnosis and/or monitoring of selected diseases.

### RELATED APPLICATIONS

This application claims the benefit of Japanese Patent Application No. 2008-188840, filed Jul. 22, 2008.

### FIELD OF THE INVENTION

The present invention relates to an analysis apparatus for analyzing samples by capillary electrophoresis, wherein the apparatus may be used in the diagnosis and/or monitoring of selected diseases.

### BACKGROUND OF THE INVENTION

Proteins such as albumin, globulin (α1, α2, β, Γ globulin), fibrinogen, and hemoglobin are contained in blood. Selected characteristics of these proteins, such as their concentrations, relative ratios, and mutations are analyzed and used for diagnosis of diseases. Among these proteins, albumin, globulin (α1, α2, β, Γ globulin), and fibrinogen are contained in blood in significant amounts. The relative ratios of these proteins are considered as important indicators in the diagnosis of disorders such as cirrhosis, nephrotic syndrome and collagen disease. These ratios may be analyzed by methods such as cellulose acetate membrane electrophoresis. Hemoglobulin (Hb) includes, for example, hemoglobin A (HbA), hemoglobin F (HbF), hemoglobin S (HbS) and glycosylated hemoglobin. Among these, HbA and HbF represent normal hemoglobin in human. HbS is an abnormal hemoglobin, in which the 6^{th} glutamic acid of the β chain has been substituted with valine, and is an indicator in the diagnosis of sickle-cell anemia. Since the glycosylated hemoglobin is hemoglobin that has reacted with glucose in blood and reflects the past history of the blood glucose level in a biological body, it may be used as an indicator in the diagnosis and treatment of diabetes. Among glycosylated hemoglobins, hemoglobin A1c (HbA1c), the β-chain N-terminal valine of which is glycosylated is an important indicator of diabetes and is typically measured in routine physical examinations. As described above, selected proteins in blood, such as hemoglobin, are important indicators of various diseases. Hence, the development of analysis apparatuses are desired which are relatively inexpensive to operate, of reduced size to accommodate limited spaces, and which are suitable for use in, for example, routine laboratory tests.

Examples of methods used to measure hemoglobin in blood include immunological methods, enzymatic methods, affinity chromatography methods, HPLC methods and capillary electrophoresis methods. Since immunological methods and enzymatic methods can be applied to autoanalysis apparatuses, they have the advantage of being capable of handling large numbers of specimens. However, immunological methods and enzymatic methods lack measurement accuracy and typically require at least about 10 minutes for sample analysis. With respect to affinity chromatography methods, measurement accuracy of HbA1c is low and requires at least about two minutes for sample analysis. HPLC methods are used widely for measuring hemoglobin (e.g., JP3429709 B). However, HPLC methods require large and expensive special apparatuses which are difficult to reduce in size, cost, and speed of analysis.

In contrast, capillary electrophoresis apparatuses can be downsized using microchips and analysis can be performed in about 90 seconds (e.g., WO 2008/047703 A1). However, from the viewpoint of routine use in laboratory tests, further reduction in analysis time is highly desired. The apparatuses of the present invention satisfy the aforementioned needs by being small in size, inexpensive to operate and of a short analysis time.

### SUMMARY OF THE INVENTION

An aspect of the invention is a method of analyzing a sample comprising applying a sample to a capillary electrophoresis analysis apparatus; and performing electrophoretic separation and detection of the sample in greater than 0 seconds but less than 35 seconds, wherein the capillary electrophoresis analysis apparatus comprises an electrophoresis chip comprising a substrate; a capillary channel; and a plurality of liquid reservoirs in communication with each other via the capillary channel; a voltage application unit comprising an electrode in communication with the capillary channel; and an absorbance measurement unit, and wherein the detection is measured by the absorbance measurement unit.

Another aspect of the invention is a method of diagnosing diabetes in a subject comprising obtaining a sample of blood from a subject; applying the sample to a capillary electrophoresis apparatus; and performing electrophoretic separation and detection of the sample for determining the amount of glycated hemoglobin in the sample, thereby determining whether the subject has diabetes, wherein the capillary electrophoresis analysis apparatus comprises an electrophoresis chip comprising a substrate; a capillary channel; and a plurality of liquid reservoirs in communication with each other via the capillary channel; a voltage application unit comprising an electrode in communication with the capillary channel; and an absorbance measurement unit, wherein the detection is measured by the absorbance measurement unit.

Another aspect of the invention is a method of monitoring diabetes in a subject comprising obtaining a sample of blood from a subject; applying the sample to a capillary electrophoresis apparatus; and performing electrophoretic separation and detection of the sample for determining the amount of glycated hemoglobin in the sample, thereby determining whether the subject has diabetes, wherein the capillary electrophoresis analysis apparatus comprises an electrophoresis chip comprising a substrate; a capillary channel; and a plurality of liquid reservoirs in communication with each other via the capillary channel; a voltage application unit comprising an electrode in communication with the capillary channel; and an absorbance measurement unit, and wherein the detection is measured by the absorbance measurement unit.

In an exemplary embodiment, the method according to claim 1, wherein the apparatus has a width of about 10 cm to about 100 cm, a depth of about 10 cm to about 100 cm and a height of about 5 cm to about 100 cm.

In an exemplary embodiment, the method according to claim 1, wherein the capillary channel is formed on the surface of the substrate or is a tube embedded in the substrate.

In an exemplary embodiment, the method according to claim 1, wherein an inner wall surface of the capillary channel is coated with a cationic layer, an anionic layer or a neutral layer.

In an exemplary embodiment, the method according to claim 1, wherein the plurality of liquid reservoirs are depressions formed on the surface of the substrate.

In an exemplary embodiment, the method according to claim 1, wherein the electrophoresis chip has a length of about 10 mm to about 100 mm, a width of about 10 mm to about 60 mm and a thickness of about 0.3 mm to about 5 mm.

In an exemplary embodiment, the method according to claim 1, wherein the capillary channel contains an electrophoresis running buffer, wherein the electrophoresis running buffer comprises a sulfated polysaccharide.

In an exemplary embodiment, the method according to claim 7, wherein the sulfated polysaccharide is a chondroitin sulfate.

In an exemplary embodiment, the method according to claim 1, wherein the capillary channel has a diameter of about 25 μm to about 100 μm and a length of about 0.5 cm to about 15 cm.

In an exemplary embodiment, the method according to claim 1, wherein the capillary channel contains a cross-sectional shape perpendicular to the channel direction.

In an exemplary embodiment, the method according to claim 10, wherein the cross-sectional shape is circular, rectangular, ellipsoidal or polygonal.

In an exemplary embodiment, the method according to claim 11, wherein when the cross-sectional shape is circular, the diameter thereof is about 25 μm to about 100 μm.

In an exemplary embodiment, the method according to claim 11, wherein when the cross-sectional shape is rectangular, the width thereof is about 25 μm to about 100 μm and the depth thereof is about 25 μm to about 100 μm.

In an exemplary embodiment, the method according to claim 1, wherein the capillary electrophoresis apparatus further comprises a pre-filter component, an air vent structure, a stray light removing unit, a position adjustment unit, a quantitative dispensing unit, a stirring unit, a liquid sending unit or combinations thereof.

In an exemplary embodiment, the method according to claim 1, wherein the electrophoresis chip surface has been treated with at least one of phosphoric acid, UV radiation, alkali dipping, an inorganic nanomicroparticle coating, graft co-polymerization and corona discharge to minimize adsorption of the sample.

In an exemplary embodiment, the method according to claim 7, wherein the electrophoresis running buffer further comprises a chaotropic anion.

In an exemplary embodiment, the method according to claim 1, wherein the sample comprises a blood protein.

In an exemplary embodiment, the method according to claim 17, wherein the blood protein comprises hemoglobin.

In an exemplary embodiment, the method according to claim 18, wherein the hemoglobin is at least one of normal hemoglobin, glycosylated hemoglobin, modified hemoglobin, variant hemoglobin, and fetal hemoglobin.

In an exemplary embodiment, the method according to claim 18, wherein the hemoglobin is at least one of hemoglobin A1c, hemoglobin F, hemoglobin A2, hemoglobin S, and hemoglobin C.

In an exemplary embodiment, the method according to claim 20, wherein the hemoglobin is hemoglobin A1c.

In an exemplary embodiment, the method according to claim 1, wherein the sample comprises hemoglobin and wherein a concentration of the hemoglobin is detected by the absorbance measurement unit.

In an exemplary embodiment, the method according to claim 22, wherein the absorbance measurement unit measures absorbance by the hemoglobin at a wavelength range of about 260 nm to about 300 nm or at a range of about 380 nm to about 450 nm.

In an exemplary embodiment, the method according to claim 1, wherein the sample comprises hemoglobin and is subjected to a hemolysis treatment.

In an exemplary embodiment, the method according to claim 24, wherein the hemolysis treatment is at least one of a surfactant treatment, an osmotic pressure treatment, and a sonication treatment.

In an exemplary embodiment, the method according to claim 1, wherein an electroosmotic flow generated during electrophoretic separation of the sample is in the range of about 3 to about 20 cm/min.

In an exemplary embodiment of the invention, the analysis apparatus is a capillary electrophoresis analysis apparatus for analyzing protein in blood by a capillary electrophoresis method that comprises an electrophoresis chip, a voltage application unit, an optional liquid sending unit, and an absorbance measurement unit, wherein the electrophoresis chip comprises a substrate, a plurality of liquid reservoirs, and a capillary channel.

In separate exemplary embodiments, the voltage application unit comprises an electrode, the plurality of liquid reservoirs is formed in the substrate, the plurality of liquid reservoirs are in communication with one another via the capillary channel, the capillary channel includes a capillary channel for sample analysis, the capillary channel for sample analysis is filled with an electrophoresis running buffer using a liquid sending unit, a sample to be applied to the apparatus comprises protein in blood, the sample is introduced into the capillary channel which is filled with the electrophoresis running buffer, the sample is subjected to an electrophoresis by applying voltage to the electrode, absorbance of the protein in blood in the sample subjected to the electrophoresis is measured by the absorbance measurement unit, and the analysis time of the protein in blood is 35 seconds or less.

In an exemplary embodiment, the analysis method of the invention provides for analysis of a sample containing a blood protein by a capillary electrophoresis method that uses an electrophoresis chip in which a capillary channel is formed and the capillary channel includes a capillary channel for sample analysis, wherein the method comprises introducing the sample into the capillary channel for sample analysis that is filled with an electrophoresis running buffer and subjecting the sample to electrophoresis by applying a voltage to an electrode; and measuring a predetermined absorbance of the blood protein in the sample, wherein the analysis time of the sample containing the blood protein is 30 seconds or less.

In an exemplary embodiment of the invention, the capillary electrophoresis analysis apparatus described herein and the analysis method using the apparatus is suitable for micro total analysis systems (μTAS).

### BRIEF DESCRIPTION OF THE DRAWINGS

The following figures illustrate particular embodiments of the invention and are not intended to limit the scope of the invention as described herein.

FIG. 1 (A) is a planar view of an example of an electrophoresis chip in the capillary electrophoresis analysis apparatus of the invention. FIG. 1 (B) is a cross-sectional view of the electrophoresis chip shown in FIG. 1 (A) represents the view of the electrophoresis chip in the direction of line I-I.

FIG. 2 is a schematic view of an example of the capillary electrophoresis analysis apparatus of the invention.

FIG. 3 (A) is a planar view of another example of an electrophoresis chip in the capillary electrophoresis analysis apparatus of the invention. FIG. 3 (B) is a cross-sectional view of the electrophoresis chip shown in FIG. 3 (A) viewed in the direction of line I-I. FIG. 3 (C) is a cross-sectional view of the electrophoresis chip shown in FIG. 3 (A) viewed in the direction of line II-II.

FIG. 4 (A) is a planar view of yet another example of an electrophoresis chip in the capillary electrophoresis analysis apparatus of the invention. FIG. 4 (B) shows a perspective view of the electrophoresis chip shown in FIG. 4 (A).

FIG. 5 is a schematic view of another example of the capillary electrophoresis analysis apparatus of the invention.

FIG. 6 is a graph of an analysis result of hemoglobin in an example of the analysis method of the invention.

FIG. 7 is a graph of an analysis result of hemoglobin in another example of the analysis method of the invention.

FIG. 8 is a graph of an analysis result of hemoglobin in yet another example of the analysis method of the present invention.

FIG. 9 is a graph showing an analysis result of hemoglobin in yet another example of the analysis method of the invention.

FIG. 10 is a graph of an analysis result of hemoglobin in yet another example of the analysis method of the invention.

FIG. 11 is a graph of an analysis result of hemoglobin in an analysis method of a comparative example.

FIG. 12 is a graph of an analysis result of hemoglobin in an analysis method of another comparative example.

### DETAILED DESCRIPTION

The capillary electrophoresis analysis apparatus of the invention is capable of rapidly and accurately analyzing a sample, such as, for example, a sample containing a blood protein, by a capillary electrophoresis method that permits for rapid and accurate analysis of the sample. The apparatus has the attributes of being of a reduced size and simplified operation, and is inexpensive to manufacture compared to other purification systems.

In an exemplary embodiment, the maximum width of the whole apparatus is in the range of about 10 cm to about 100 cm, such as about 15 cm to about 85 cm, such as about 20 cm to about 75 cm, such as about 25 cm to about 65 cm, such as about 30 cm to about 55 cm, such as about 35 cm to about 45 cm. In an exemplary embodiment, the maximum depth of the whole apparatus is in the range of about 10 cm to about 100 cm, such as about 15 cm to about 85 cm, such as about 20 cm to about 75 cm, such as about 25 cm to about 65 cm, such as about 30 cm to about 55 cm, such as about 35 cm to about 45 cm. In an exemplary embodiment, the maximum height of the whole apparatus is in the range of about 5 cm to about 100 cm, such as about 10 cm to about 85 cm, such as about 15 cm to about 75 cm, such as about 20 cm to about 65 cm, such as about 30 cm to about 55 cm, such as about 35 cm to about 45 cm.

In an exemplary embodiment, in the capillary channel for sample analysis, a cross-sectional shape perpendicular to a channel direction is circular or rectangular in configuration. In an embodiment where the configuration is circular, a diameter thereof is in a range of about 25 μm to about 100 μm. In an embodiment where the configuration is rectangular, a width thereof is in a range of about 25 μm to about 100 μm and a depth thereof is in a range of about 25 μm to about 100 μm. In an exemplary embodiment, electrophoresis of the sample begins from an electrophoresis starting point, the absorbance of the blood protein in the sample subjected to the electrophoresis is measured at a detecting point, and a distance from the electrophoresis starting point to the detecting point is 5 cm or less.

In an exemplary embodiment, a capillary electrophoresis analysis apparatus of the invention further comprises a pre-filter component for removal of any undesired foreign materials present in the sample to be analyzed. In an exemplary embodiment, the foreign materials range in size from about 1 μm to about 5 μm. In an exemplary embodiment, the foreign materials include cell membrane fragments, plasma proteins and lipids derived from blood cells. The type and size of the filter is not limiting as long as it is able to remove the undesired materials that could potentially interfere with effective separation and analysis of a sample. In an exemplary embodiment, the filter may be derived from a metal (e.g., titanium or stainless steel), a resin (e.g., polyethylene, PEEK, polypropylene, polyethylene terphthalate, nylon, rayon, acrylic, vinylidene chloride or Teflon™), cotton, wool, coconut fiber, hemp or glass fiber. In an exemplary embodiment, use of the pre-filter component does not result in a significant increase in pressure across the filter. In an exemplary embodiment, the diameter of the filter is from about 0.1 to about 10 mm, such as about 0.5 mm to about 8 mm. In an exemplary embodiment, the thickness of the filter is from about 0.1 mm to about 5 mm, such as about 0.2 mm to about 3 mm. In an exemplary embodiment, the diameter of the filtration pore is from about 0.1 μm to about 5 μm, such as about 0.2 μm to about 3 μm. It is an objective to maintain an acceptable void ratio. The profile of the filter is not limiting as long as the filter has a structure which does not disturb the fluid flow. In exemplary embodiments, the profile is conical, columnar, circular truncated cones or two cones wherein the bottoms of the cones are in contact with each other.

In an exemplary embodiment, a capillary electrophoresis analysis apparatus of the invention further comprises an air vent structure for venting the air that enters into the flow path of the apparatus. The positioning and size of the air vent structure is not limiting as long as air in the flow path can be effectively removed. In an exemplary embodiment, the pore diameter for air venting ranges from about 0.01 mm to about 3 mm. In an exemplary embodiment, the air vent structure contains Teflon™.

In an exemplary embodiment, a capillary electrophoresis analysis apparatus of the invention further comprises a stray light removing unit. Because the absorbance measurement accuracy is further improved by including a stray light removing unit, a more accurate measurement can be performed. In the present invention, stray light refers to light that is not contributing to detection of the transmitted light. The stray light removing unit is not particularly limited, and may include, for example an aperture, a slit or a pinhole which are arranged between the light source and the capillary channel for sample analysis. The shape of a hole of the aperture, the slit, and the pinhole is not particularly limited, and may, for example, include circular or rectangular. In exemplary embodiments where the shape of the hole of the aperture, the slit or the pinhole is circular, the diameter thereof may be in the same range as the inner diameter of the capillary channel. In exemplary embodiments where the shape of the hole of the aperture, the slit or the pinhole is rectangular, the length in the short side direction of the hole is may also be in the same range as the inner diameter of the capillary channel.

In an exemplary embodiment, a capillary electrophoresis analysis apparatus of the invention further comprises a position adjustment unit, wherein at least one of a position of the electrophoresis chip and a position of the absorbance measurement unit is capable of adjustment by the position adjustment unit.

In an exemplary embodiment, a capillary electrophoresis analysis apparatus of the invention further comprises a buffer solution and optionally a diluent.

In an exemplary embodiment, a capillary electrophoresis analysis apparatus of the invention further comprises a chip surface that has been treated with at least one of phosphoric acid, UV radiation, alkali dipping, an inorganic nanomicroparticle coating, graft co-polymerization and corona discharge as a means of suppressing undesired adsorption of a sample onto surfaces including, but not limited to, reservoir surfaces and capillary channel surfaces.

In an exemplary embodiment, a capillary electrophoresis analysis apparatus of the invention further includes a quantitative dispensing unit. Because the quantitative dispensing of a sample or reagent can be performed automatically by means of a quantitative dispensing unit, measurements can be performed with minimum effort. In an exemplary embodiment, the quantitative dispensing unit is provided in the electrophoresis chip or alternatively, outside of the electrophoresis chip.

Examples of quantitative dispensing units include, but are not limited to, a measurement channel. The measurement channel is not particularly limited and may be a part of the capillary channel of the electrophoresis chip. The measurement channel can pool or retain a certain amount of sample or reagent such as an electrophoresis running buffer. Examples of the measurement channel include, but are not limited to, a measurement channel for the sample and a measurement channel for the electrophoresis running buffer. The quantitative dispensing unit may optionally contain a suction and discharge mechanism.

In an exemplary embodiment, a capillary electrophoresis analysis apparatus of the invention further includes a stirring unit. Because a solution such as a sample, a reagent, and the like, can be mixed automatically by means of a stirring unit, a measurement can be performed simply. The stirring unit is not particularly limited and may include a stir bar. The stir bar is not particularly limited and may include a small piece of a ferromagnet whose surface is sealed with, for example, polytetrafluoroethylene. A solution in the liquid reservoir can be stirred, for example, by disposing the stir bar in a mixing liquid reservoir for mixing a sample and a reagent, and providing an electromagnetic stirring machine such as a magnetic stirrer at a bottom surface of the liquid reservoir. Alternatively, for example, the quantitative dispensing unit may serve as the quantitative dispensing unit and the stirring unit. For example, the aforementioned two solutions can be stirred by means of a quantitative dispensing unit, for example, by suction and discharging a mixture of a sample and an electrophoresis running buffer.

In an exemplary embodiment, a capillary electrophoresis analysis apparatus of the invention further include a liquid sending unit for introducing a solution (e.g., a solution containing sample) into the capillary channel. Because an electrophoresis chip can be filled automatically or introduced with a solution by means of a liquid sending unit, a measurement can be performed with minimum effort. By means of the liquid sending unit, a reagent such as an electrophoresis running buffer, an analytical reagent, a diluent, a washing liquid and a sample can be efficiently introduced into the capillary channel. The liquid sending unit is not particularly limited, and may include, for example, a suction unit, a discharge unit, and/or a voltage application unit.

In an exemplary embodiment, the suction unit (vacuum unit) is provided with a vacuum pump and a drain portion. The drain portion may, for example, be disposed at one end of the capillary channel, and the vacuum pump may be connected to the drain portion. By reducing the pressure in the capillary channel with the vacuum pump via the drain, the solution can be suctioned up and introduced into the capillary channel from the other end of the channel.

The discharge unit (pressure unit) may be provided, for example, with a pressure pump and a drain portion. The drain portion may, for example, be disposed at one end of the capillary channel and the pressure pump may be connected to the drain portion. By applying pressure to the inside of the channel by discharging air thereinto with the pressure pump via the drain, the solution can be introduced into the capillary channel by discharging air from the end of the channel.

In the capillary electrophoresis analysis apparatus of the invention, an inner wall surface of the capillary channel for sample analysis may have an ionic functional group. In an exemplary embodiment, the pH of the electrophoresis running buffer may be in a range of about 4.0 to about 6.0, and an electroosmotic flow generated at the time of voltage application may be about 3 cm/min or more.

In an exemplary embodiment, the electrophoresis running buffer contains a sulfated polysaccharide.

In a particular embodiment, the sulfated polysaccharide is chondroitin sulfate.

In an exemplary embodiment, in the capillary channel for sample analysis, the cross-sectional shape is rectangular and has a width and a depth of about 30 μm each, the distance from the electrophoresis starting point to the detecting point is at least about 0.25 cm and less than about 0.75 cm, and an electric field for separation is about 300 V/cm or less.

In another exemplary embodiment where the cross-sectional shape is rectangular and has a width and a depth of about 30 μm each, the distance from the electrophoresis starting point to the detecting point is at least about 0.75 cm and less than about 1.25 cm, and the electric field for separation is in a range of about 300 to about 600 V/cm.

In another exemplary embodiment where the cross-sectional shape is rectangular and has a width and a depth of about 30 μm each, the distance from the electrophoresis starting point to the detecting point is at least about 1.25 cm and less than about 1.75 cm, and the electric field for separation is in a range of about 400 to about 650 V/cm. In another exemplary embodiment where the cross-sectional shape is rectangular and has a width and a depth of about 30 μm each, the distance from the electrophoresis starting point to the detecting point is at least about 1.75 cm and less than about 2.25 cm, and the electric field for separation is in a range of about 450 to about 700 V/cm.

In an exemplary embodiment in the capillary channel for sample analysis, the cross-sectional shape is rectangular and has a width and a depth of about 40 μm each, the distance from the electrophoresis starting point to the detecting point is at least about 0.25 cm and less than about 0.75 cm, and an electric field for separation is about 250 V/cm or less. In another exemplary embodiment where the cross-sectional shape is rectangular and has a width and a depth of about 40 μm each, the distance from the electrophoresis starting point to the detecting point is at least about 0.75 cm and less than about 1.25 cm, and the electric field for separation is in a range of about 250 to about 500 V/cm. In another exemplary embodiment where the cross-sectional shape is rectangular and has a width and a depth of about 40 μm each, the distance from the electrophoresis starting point to the detecting point is at least about 1.25 cm and less than about 1.75 cm, and the electric field for separation is in a range of about 375 to about 550 V/cm. In another exemplary embodiment where the cross-sectional shape is rectangular and has a width and a depth of about 40 μm each, the distance from the electrophoresis starting point to the detecting point is at least about 1.75 cm and less than about 2.25 cm, and the electric field for separation is in a range of about 400 to about 600 V/cm.

In an exemplary embodiment, in the capillary channel for sample analysis, the cross-sectional shape is rectangular and has a width and a depth of about 50 μm each, the distance from the electrophoresis starting point to the detecting point is at least about 0.25 cm and less than about 0.75 cm, and an electric field for separation is about 200 V/cm or less.

In another exemplary embodiment where the cross-sectional shape is rectangular and has a width and a depth of about 50 μm each, the distance from the electrophoresis starting point to the detecting point is at least about 0.75 cm and less than 1.25 cm, and the electric field for separation is in a range of about 200 to about 450 V/cm.

In another exemplary embodiment where the cross-sectional shape is rectangular and has a width and a depth of about 50 μm each, the distance from the electrophoresis starting point to the detecting point is at least about 1.25 cm and less than about 1.75 cm, and the electric field for separation is in a range of about 300 to about 500 V/cm.

In another exemplary embodiment where the cross-sectional shape is rectangular and has a width and a depth of about 50 μm each, the distance from the electrophoresis starting point to the detecting point is at least about 1.75 cm and less than 2.25 cm, and the electric field for separation is in a range of about 350 to about 550 V/cm.

In an exemplary embodiment, in the capillary channel for sample analysis, the cross-sectional shape is rectangular and has a width and a depth of about 60 μm each, the distance from the electrophoresis starting point to the detecting point is at least about 0.25 cm and less than 0.75 cm, and an electric field for separation is about 150 V/cm or less.

In another exemplary embodiment where the cross-sectional shape is rectangular and has a width and a depth of about 60 μm each, the distance from the electrophoresis starting point to the detecting point is at least about 0.75 cm and less than about 1.25 cm, and the electric field for separation is in a range of about 150 to about 400 V/cm.

In another exemplary embodiment where the cross-sectional shape is rectangular and has a width and a depth of about 60 μm each, the distance from the electrophoresis starting point to the detecting point is at least about 1.25 cm and less than about 1.75 cm, and the electric field for separation is in a range of about 250 to about 450 V/cm.

In another exemplary embodiment where the cross-sectional shape is rectangular and has a width and a depth of about 60 μm each, the distance from the electrophoresis starting point to the detecting point is at least about 1.75 cm and less than about 2.25 cm, and the electric field for separation is in a range of about 300 to about 500 V/cm.

In an exemplary embodiment, the sample for analysis contains a blood protein. In a particular embodiment, the blood protein is hemoglobin.

In a particular embodiment, the hemoglobin is at least one of hemoglobin A1c (HbA1c) and hemoglobin F (HbF).

In a particular embodiment, the sample to be analyzed on the capillary electrophoresis apparatus of the invention contains hemoglobin A1c (HbA1c), and a hemoglobin A1c concentration (HbA1c concentration) is calculated as a result of the analysis of the hemoglobin A1c (HbA1c).

In an exemplary embodiment, the sample is prepared by subjecting blood to a hemolysis treatment, and the hemoglobin in the sample that is prepared by subjecting the blood to the hemolysis treatment is analyzed.

In an exemplary embodiment, in the capillary channel for sample analysis, a cross-sectional shape perpendicular to a channel direction is circular or rectangular. In a particular embodiment, the cross-section shape is circular, and the diameter thereof is in a range of about 25 μm to about 100 μm. In a particular embodiment, the cross-section shape is rectangular, and the width thereof is in a range of about 25 μm to about 100 μm and the depth thereof is in a range of about 25 μm to about 100 μm. In an exemplary embodiment, the electrophoresis of the sample begins from an electrophoresis starting point, the absorbance of the sample, wherein the sample contains a blood protein and is subjected to the electrophoresis, is measured at a detecting point, and the distance from the electrophoresis starting point to the detecting point is about 5 cm or less.

In an exemplary embodiment, an inner wall surface of the capillary channel for sample analysis may have an ionic functional group. In an exemplary embodiment, the pH of the electrophoresis running buffer may be in a range of about 4.0 to about 6.0, and an electroosmotic flow generated at the time of voltage application is at least 3 cm/min.

Hereinafter, the capillary electrophoresis analysis apparatus of the present invention is described in detail.

As described herein, the capillary electrophoresis analysis apparatus of the invention comprises an electrophoresis chip, a voltage application unit, an optional liquid sending unit, and an absorbance measurement unit, and other configurations not particularly limited.

In an exemplary embodiment, the electrophoresis chip of the capillary electrophoresis analysis apparatus comprises a substrate, a plurality of liquid reservoirs, and a capillary channel.

In an exemplary embodiment, the maximum length of the chip is in the range of about 10 mm to about 100 mm, such as about 15 mm to about 85 mm, such as about 20 mm to about 75 mm, such as about 25 mm to about 65 mm, such as about 30 mm to about 55 mm, such as about 35 mm to about 45 mm. In an exemplary embodiment, the maximum width of the chip is in the range of about 10 mm to about 60 mm, such as about 15 mm to about 55 mm, such as about 20 mm to about 50 mm, such as about 25 mm to about 45 mm, such as about 30 mm to about 40 mm. In an exemplary embodiment, the maximum thickness of the chip is in the range of about 0.3 mm to about 5 mm, such as about 0.5 mm to about 4 mm, such as about 0.7 mm to about 3 mm, such as about 1 mm to about 2 mm.

In an exemplary embodiment, the maximum length of the electrophoresis chip is the length of the portion that is longest in the longitudinal direction of the electrophoresis chip. In an exemplary embodiment, the maximum width of the electrophoresis chip is the length of the portion that is longest in the short side direction of the electrophoresis chip. In an exemplary embodiment, the maximum thickness of the electrophoresis chip is the length of the portion that is longest along the direction (thickness direction) perpendicular to both the longitudinal direction and the short side direction of the electrophoresis chip.

In exemplary embodiments, the electrophoresis chip further comprises a blood collection mechanism or an electrophoresis chip combined with a lancet.

In an exemplary embodiment, the electrophoresis chip comprises a substrate, a plurality of liquid reservoirs, and a capillary channel.

In an exemplary embodiment, the maximum length of the electrophoresis chip is in the range of about 10 mm to about 100 mm, such as about 15 mm to about 80 mm, such as about 20 mm to about 60 mm, such as about 30 mm to about 50 mm.

In an exemplary embodiment, the maximum width of the electrophoresis chip is in the range of about 10 mm to about 60 mm, such as about 15 to about 50 mm, such as about 20 mm to about 40 mm, such as about 25 mm to about 35 mm.

In an exemplary embodiment, the maximum thickness of the electrophoresis chip is in the range of about 0.3 mm to about 5 mm, such as about 0.5 mm to about 3 mm, such as about 1 mm to about 2 mm.

In an exemplary embodiment, the maximum length of the electrophoresis chip is in the range of about 30 mm to about 70 mm, such as about 35 mm to about 60 mm, such as about 40 mm to about 55 mm.

In an exemplary embodiment, the maximum length of the electrophoresis chip is the length of the portion that is longest in the longitudinal direction of the electrophoresis chip. In an exemplary embodiment, the maximum width of the electrophoresis chip is the length of the portion that is longest in the short side direction of the electrophoresis chip. In an exemplary embodiment, the maximum thickness of the electrophoresis chip is the length of the portion that is longest in the direction (thickness direction) perpendicular to both the longitudinal direction and the short side direction of the electrophoresis chip.

In exemplary embodiments, the substrate is composed of one piece of substrate, or an upper substrate and a lower substrate laminated together. In exemplary embodiments, the substrate is a glass material or a polymeric material. The glass material is not particularly limited, and examples thereof include synthetic silica glass, borosilicate glass, fused silica, etc. The polymeric material is not particularly limited, and examples thereof include polymethylmethacrylate (PMMA), cycloolefin polymer (COP), polycarbonate (PC), polydimethylsiloxane (PDMS), polystyrene (PS), polylactic acid (PLA), etc.

In an exemplary embodiment, the liquid reservoir contains a concave (depressed) portion provided in the substrate and a space portion provided in the substrate. In an exemplary embodiment, the concave portion is formed in the thickness direction of the substrate. In an exemplary embodiment, an upper substrate and a lower substrate are provided as described herein, wherein one of the substrates, in which a through-hole is provided, may be laminated onto the other substrate. The reservoir is then formed by laminating a bottom part of a through-hole formed in an upper substrate with a lower substrate. The form of the liquid reservoir is not particularly limited and examples thereof include, but are not limited to, a quadrangular prism, a quadrangular pyramid and a cone. Further, the volume of each liquid reservoir is not particularly restricted and may be, for example, in the range of about 1 mm³ to about 1000 mm³, such as in the range of about 5 mm³ to about 800 mm³, such as about 10 mm³ to about 600 mm³, such as about 10 mm³ to about 100 mm³, such as about 20 mm³ to about 500 mm³, such as about 30 mm³ to about 400 mm³, such as about 50 mm³ to about 300 mm³, such as about 75 mm³ to about 200 mm³, such as about 85 mm³ to about 150 mm³. The volume of each of the liquid reservoirs may all be the same or each may be different. The volume of each liquid reservoir is not particularly limited and may all be the same or may each be different.

The capillary channel may be formed in the substrate or may be a capillary tube embedded in the substrate.

In the capillary channel, the cross-sectional shape perpendicular to a channel direction includes, but is not limited to, circular, rectangular or ellipsoidal. In an exemplary embodiment, the cross-sectional shape of the capillary channel is circular and the diameter thereof is in the range of about 1000 μm to about 1000 μm, such as about 10 μm to about 200 μm, such as about 25 μm to about 100 μm. In an exemplary embodiment, the cross-sectional shape of the capillary channel is rectangular and the width thereof is in the range of about 10 μm to about 200 μm, the depth thereof is in the range of about 10 μm to about 200 μm, and the length thereof is in the range of about 0.5 cm to about 15 cm, such as about 1 cm to about 5 cm.

As described above, the capillary channel may be formed by the substrate or may be formed in the substrate by embedding a capillary tube therein. In the former case, the material of the capillary channel is, for example, the material of the substrate. In the latter case, the material of the capillary channel is, for example, the material of the embedded capillary tube. In an exemplary embodiment, the material of the capillary channel is, for example, but not limited to, a glass material such as synthetic silica glass, borosilicate glass, or fused silica polymeric material such as polymethylmethacrylate (PMMA), cycloolefin polymer (COP), polycarbonate (PC), polydimethylsiloxane (PDMS), polystyrene (PS), polylactic acid (PLA), polyethylene (PE), polytetrafluoroethylene (PTFE), or polyetheretherketone (PEEK). A commercially available product may be also used as the capillary tube.

Normally, an inner wall of a glass capillary channel is negatively charged. A glass capillary channel can be made to have a positive charge at the inner wall surface, however, by introducing a cationic group. For a capillary channel that is polymeric in composition, the presence or absence of polar groups in the polymer and the types of polar groups typically determine whether an inner wall of the capillary channel is positively or negatively charged or charge-free (nonpolar). A charge-free polymer capillary channel can be converted to a polymer capillary that is charged at the inner wall surface by introducing an anionic or cationic polar group.

In an exemplary embodiment, the inner wall of the capillary channel for sample analysis contains an ionic functional group at the surface thereof, wherein the ionic functional group is a cationic group or an anionic group.

In an exemplary embodiment, the capillary channel for sample analysis has a cationic group at the inner wall surface which may be formed by coating the inner wall of the capillary channel with a cationic group-containing compound. Hereinafter, a coating of cationic group-containing compound may be referred to as a cationic layer. Due to the coating of the cationic group-containing compound (the cationic layer), the inner wall of the capillary channel for sample analysis is prevented from adsorbing at least a portion of the sample. Further, due to the coating of the cationic group-containing compound, the electroosmotic flow tends to be faster compared to an untreated (uncoated) capillary channel. The cationic group-containing compound is not particularly limited and examples thereof include, for example, a compound containing the cationic group and a reactive group. For example, in a case where the capillary channel for sample analysis is made of glass or fused silica, a compound (such as a silylation agent) containing a cationic group and silicon can be used as the compound containing a cationic group and a reactive group. In an exemplary embodiment, the cationic group includes, but is not limited to, an amino group and an ammonium group. In a particular embodiment, the cationic group-containing compound includes a silylation agent having at least one cationic group, wherein the cationic group is an amino group or an ammonium group. The amino group may be a primary amino group, a secondary amino group, or a tertiary amino group or salts thereof.

Examples of the silylation agent with the cationic group include, but are not limited to, N-(2-diaminoethyl)-3-propyltrimethoxysilane, aminophenoxydimethylvinylsilane, 3-aminopropyldiisopropylethoxysilane, 3-aminopropylmethylbis(trimethylsiloxy)silane, 3-aminopropylpentamethyldisiloxane, 3-aminopropylsilanetriol, bis(p-aminophenoxy)dimethylsilane, 1,3-bis(3-aminopropyl)tetramethyldisiloxane, bis(dimethylamino)dimethylsilane, bis(dimethylamino)vinylmethylsilane, bis(2-hydroxyethyl)-3-aminopropyltriethoxysilane, 3-cyanopropyl(diisopropyl)dimethylaminosilane, (aminoethylaminomethyl)phenethyltrimethoxysilane, N-methylaminopropyltriethoxysilane, tetrakis(diethylamino)silane tris(dimethylamino)chlorosilane, and tris(dimethylamino)silane.

With respect to the cationic group-containing compound, a silicon atom in the silylation agent may be substituted by, for example, titanium or zirconium. One cationic group-containing compound may be used or two or more cationic group-containing compounds may be used in combination.

In an exemplary embodiment, the coating of the inner wall of the capillary channel for sample analysis using a silylation agent is carried out as follows. First, a treatment solution is prepared by dissolving or dispersing the silylation agent in an organic solvent. Dichloromethane, toluene, methanol and acetone are suitable as the organic solvent used for preparing the treatment solution. The concentration of the silylation agent in the treatment solution is not particularly limited. The treatment solution is passed through a capillary channel made of glass or fused silica, and heated. As a result of this heating, the silylation agent containing the cationic group is covalently-bonded to the inner wall of the capillary channel. Thereafter, as an after-treatment, the residual organic solvent remaining in the capillary channel is washed away with at least one of an acid solution such as phosphoric acid; an alkaline solution; and a surfactant solution. This washing is preferably performed, although it is optional. A commercially-available product can be used as the capillary channel, the inner wall of which is coated with the silylation agent.

A capillary channel for sample analysis having an anionic group at the inner wall surface thereof may be formed by coating the inner wall of the capillary channel with an anionic group-containing compound. Hereinafter, the coating of an anionic group-containing compound may be referred to as an anionic layer. Due to the coating of the anionic group-containing compound (the anionic layer), the inner wall of the capillary channel for sample analysis is prevented from adsorbing at least a portion of the sample, more particularly, for example, a negatively-charged protein or the like in a sample. Further, due to the coating of an anionic group-containing compound, the electroosmotic flow tends to be faster compared to an untreated (uncoated) capillary channel.

The direction of the electroosmotic flow is opposite in the case of an inner wall coating of an anionic group-containing compound compared to the case of an inner wall coating of a cationic group-containing compound. In an exemplary embodiment, the sample and the anionic group-containing compound form a complex. When the complex is subjected to an electrophoresis, the separation efficiency is increased relative to the case where the uncomplexed sample is independently subjected to electrophoresis. As a result, faster and more accurate analyses can be performed. In an exemplary embodiment, the anionic group-containing compound which forms a complex with the sample is an anionic group-containing polysaccharide. In an exemplary embodiment, the anionic group-containing compound which complexes with the sample is present in the electrophoresis running buffer.

Examples of the anionic group-containing polysaccharide include, but are not limited to, a sulfated polysaccharide, a carboxylated polysaccharide, and a phosphorylated polysaccharide. In an exemplary embodiment, the anionic group-containing compound is a sulfated polysaccharide or a carboxylated polysaccharide. Sulfated polysaccharides include, but are not limited to, chondroitin sulfate and heparin. In a particular embodiment, the sulfated polysaccharide is chondroitin sulfate such as chondroitin sulfate A, chondroitin sulfate B, chondroitin sulfate C, chondroitin sulfate D, chondroitin sulfate E, chondroitin sulfate H, and chondroitin sulfate K. In a particular embodiment, the chondroitin sulfate is chondroitin sulfate C. Carboxylated polysaccharides include, but are not limited to, algin acid or its salt (such as sodium alginate).

An anionic layer may be laminated onto the inner wall of the capillary channel for sample analysis via an intercalated layer. In an exemplary embodiment, the intercalated layer is formed using a cationic group-containing compound. For example, the anionic layer laminated via the intercalated layer may be formed by coating the inner wall of the capillary channel for sample analysis with a cationic group-containing compound and then contacting the cationic coated layer with a liquid containing an anionic group-containing compound. The liquid that forms the anionic group-containing compound may be separately prepared. From the viewpoint of operation efficiency, it is often preferable that an electrophoresis running buffer containing the anionic group-containing compound is passed through the capillary channel, resulting in formation of the intercalated layer on the inner wall of the capillary channel.

The invention is not limited to the above-described coatings on the inner wall of only the capillary channel for sample analysis. Rather, the inner walls of other capillary channels formed on the substrate may also have their inner walls coated with ionic or nonionic functional groups. The coatings on the inner walls of these other capillary channels may be formed in the same manner as the above described coatings on the inner walls of the capillary channel for sample analysis.

In an exemplary embodiment, a sample containing a blood protein to be analyzed and an electrophoresis running buffer are introduced into the capillary channel for sample analysis.

In an exemplary embodiment, the electrophoresis running buffer contains an organic acid. Examples of the organic acid include, but are not limited to, maleic acid, tartaric acid, succinic acid, fumaric acid, phthalic acid, malonic acid and malic acid. In another exemplary embodiment, the electrophoresis running buffer contains a weak base. Examples of the weak base include, but are not limited to, arginine, lysine, histidine and tris. Examples of the electrophoresis running buffer include, but are not limited to, N-(2-acetamido)iminodiacetic acid (ADA) buffer solution, morpholinoethanesulfonic acid (MES) buffer solution, bis(2-hydroxyethyl)iminotris(hydroxymethyl)methane (Bis-Tris) buffer solution, piperazine-1,4-bis(2-ethanesulfonic acid) (PIPES) buffer solution, N-(2-acetamido)-2-aminoethanesulfonic acid (ACES) buffer solution, 2-Hydroxy-3-morpholinopropanesulfonic acid (MOPSO) buffer solution, N,N-bis(2-hydroxyethyl)-2-aminoethanesulfonic acid (BES) buffer solution, 2-[4-(2-hydroxyethyl)-1-piperazinyl]ethanesulfonic acid (HEPES) buffer solution, N-tris(hydroxymethyl)methyl-2-aminoethanesulfonic acid (TES) buffer solution, and phosphoric acid buffer solution. In an exemplary embodiment, the pH of the electrophoresis running buffer is in the range of about 4.5 to about 6.0.

In an exemplary embodiment, the anionic group-containing compound is added to the electrophoresis running buffer. In an exemplary embodiment, the concentration of the anionic group-containing compound contained in the electrophoresis running buffer is in the range of about 0.001 to about 10 wt %, such as about 0.1 to about 5 wt %.

In an exemplary embodiment, a surfactant is added to the electrophoresis running buffer. Examples of the surfactant include, but are not limited to, a betaine type ampholytic surfactant and a nonionic surfactant. Examples of the betaine type ampholytic surfactant include, but are not limited to a carboxybetaine type surfactant and a sulfobetaine type surfactant. Examples of the carboxybetaine type surfactant include, but are not limited to, N,N-dimethyl-N-alkyl-N-carboxy alkylene ammonium betaine. Examples of the sulfobetaine type surfactant include, but are not limited to, N,N,N-trialkyl-N-sulfoalkyleneammonium betaine. In a particular embodiment, the sulfobetaine type surfactant is palmityl sulfobetaine.

In an exemplary embodiment, an anionic chaotropic ion may be added to the electrophoresis running buffer. The anionic chaotropic ion includes an anionic chaotropic ion (i.e., in a salt form) as well as a substance that generates an anionic chaotropic ion by ionization, (e.g., a neutral precursor compound). Examples of the salt include an acid salt, a neutral salt, and a basic salt. Examples of the anionic chaotropic ion include, but are not limited to, perchloric acid, thiocyanic acid, potassium iodide, potassium bromide, trichloroacetic acid, and trifluoroacetic acid. In an exemplary embodiment, the concentration of the anionic chaotropic ion is in the range of about 1 to about 3000 mmol/L, such as about 5 to about 100 mmol/L, such as about 10 to about 50 mmol/L.

In an exemplary embodiment, the sample contains a blood protein to be analyzed, wherein the sample is whole blood. In a particular embodiment, the whole blood to be analyzed is subjected to a hemolysis treatment. In an exemplary embodiment, the hemolysis treatment includes, but is not limited to, a surfactant treatment, an osmotic pressure treatment, a sonication treatment, a freeze/thaw treatment, a pressure treatment. In a particular embodiment, hemolysis treatment is a surfactant treatment, an osmotic pressure treatment, or a sonication treatment.

In an exemplary embodiment, the surfactant treatment is a treatment in which whole blood is hemolyzed with a diluent to which a surfactant is added. Examples of the surfactant include, but are not limited to, the aforementioned surfactants, saponin, etc. In an exemplary embodiment, the osmotic pressure treatment is a treatment in which whole blood is hemolyzed with a solution that is adjusted to have low osmotic pressure. In an exemplary embodiment, the solution includes, but is not limited to, distilled water or a diluent that is adjusted to have low osmotic pressure. In a particular embodiment, the solution is distilled water. In an exemplary embodiment, the sonication treatment is an ultrasonic processor. In an exemplary embodiment, the diluent includes, but is not limited to, distilled water and the aforementioned electrophoresis running buffer.

In an exemplary embodiment, the blood protein includes, but is not limited to, hemoglobin (Hb), albumin (Alb), globulin (α1, α2, β, Γ globulin) and fibrinogen.

Examples of the hemoglobin include, but are not limited to, normal hemoglobin (HbA0), glycosylated hemoglobin, modified hemoglobin, and fetal hemoglobin (HbF). Examples of the glycosylated hemoglobin include, but are not limited to, hemoglobin Ala (HbA1a), hemoglobin A1b (HbA1b), hemoglobin A1c (HbA1c) and GHbLys. Examples of the hemoglobin A1c include, but are not limited to, stable HbA1c and unstable HbA1c. Examples of the modified hemoglobin include, but are not limited to, carbamoylated Hb and acetylated Hb.

In an exemplary embodiment, the blood protein is an analysis item. Examples include, but are not limited to, a ratio of various hemoglobins, a hemoglobin A1c concentration, an albumin concentration, a globulin concentration, and an albumin/globulin ratio.

In an exemplary embodiment, electrophoresis of the sample is performed from an electrophoresis starting point toward a detecting point via an electroosmotic flow generated by application of voltage to an electrode. Then, at the detecting point on the capillary channel for sample analysis, a predetermined absorbance of a blood protein in the sample that is subjected to the electrophoresis is measured.

In an exemplary embodiment, the electrophoretic starting point is a point, which is placed on the capillary channel for sample analysis, where an electrophoresis of the sample that is introduced into the capillary channel for sample analysis is started upon application of a voltage. Examples of the electrophoresis starting point include, but are not limited to, a boundary between a liquid reservoir, into which the sample is introduced, and the capillary channel for sample analysis.

In an exemplary embodiment of the invention, the detecting point is a point where an absorbance of a blood protein in the sample that is subjected to the electrophoresis in the capillary channel for sample analysis is measured. The detecting point is typically placed on the capillary channel for sample analysis and the distance from the electrophoresis starting point is preferably in the range that is described herein as an exemplary embodiment.

In an exemplary embodiment, the distance from the electrophoretic starting point to the detecting point (separation length) is, in the range of about 0.5 cm to about 15 cm, such as about 1 cm to about 5 cm.

In an exemplary embodiment, the voltage is in the range of about 0.075 kV to about 20 kV, such as about 0.3 kV to about 5 kV, such as about 0.5 kV to about 4 kV, such as about 1 kV to about 3 kV. In an exemplary embodiment, the electroosmotic flow is in the range of about 3 cm/min to about 15 cm/min, such as about 8 cm/min to about 12 cm/min.

In an exemplary embodiment, the absorbance is monitored by measuring a wavelength is in the range of about 260 nm to about 300 nm or about 380 nm to about 450 nm, such as in the range of about 400 nm to about 430 nm.

In an exemplary embodiment, the analysis time of the blood protein that is contained in the sample to be analyzed is the time required for separating the blood protein and completing the detection thereof. In a particular embodiment, the analysis time of the blood protein is the time from the start of the voltage application to the electrode to the completion of detection of all proteins in the blood to be analyzed. In an exemplary embodiment, the analysis time of the blood protein exceeds 0 second and is 35 seconds or less, such as 30 seconds or less, such as 25 seconds or less, such as 20 seconds or less, such as 15 seconds or less, such as 10 seconds or less.

In an exemplary embodiment, the distance from the electrophoresis starting point to the detecting point and the electric field for separation are set in the aforementioned predetermined range in accordance with the width and the depth of the capillary channel. In an exemplary embodiment, the electric field for separation is a voltage to be applied to an interelectrode distance per cm. By setting the distance from the electrophoresis starting point to the detecting point and the electric field for separation in the aforementioned predetermined range, a sample, such as the exemplary embodiment of a blood protein, can be analyzed in a short time such as 30 seconds or less. The predetermined range is not limited to the aforementioned range, and can be adjusted suitably according to the various types of electrophoresis running buffer solutions that are chosen and also the coating of the inner wall of the capillary channel as described herein.

In various exemplary embodiments, the capillary electrophoresis analysis apparatus may further comprise at least one of a quantitative dispensing unit, a stirring unit, a stray light removing unit and a position adjustment unit.

In an exemplary embodiment, the invention provides collecting human blood from a patient and assaying the blood to determine the level of glycated hemoglobin in the blood sample through the use of the capillary electrophoresis analysis apparatus of the invention to diagnose and monitor diabetes. For example, a blood sample may be collected and applied to the capillary electrophoresis analysis apparatus and separated into its components. The amount of glycosylated Hb is detected and analyzed for determining its percentage.

Glycosylated hemoglobin has been recommended for both checking blood sugar control in people who might be pre-diabetic and for monitoring blood sugar control in patients with more elevated levels, termed diabetes mellitus. The amount of glycosylated hemoglobin provides a way to monitor diabetes because it provides information as to whether a patient's diabetes is under control. As a reference, a non-diabetic or normal subject has less than 6% HbA1C in his blood, and a patient having less than 6% HbA1C in his blood indicates that his diabetes is under control.

The capillary electrophoresis analysis apparatus of the invention may be used to diagnose and monitor diabetes in a patient. For example, the apparatus may be used to diagnose whether a subject may develop diabetes or may have diabetes. The apparatus may also be used to determine the level of glycated hemoglobin in a patient for monitoring the progression of diabetes.

The capillary electrophoresis analysis apparatus of the invention and the analysis method of the invention are illustrated by the following non-limiting examples.

### Embodiment 1

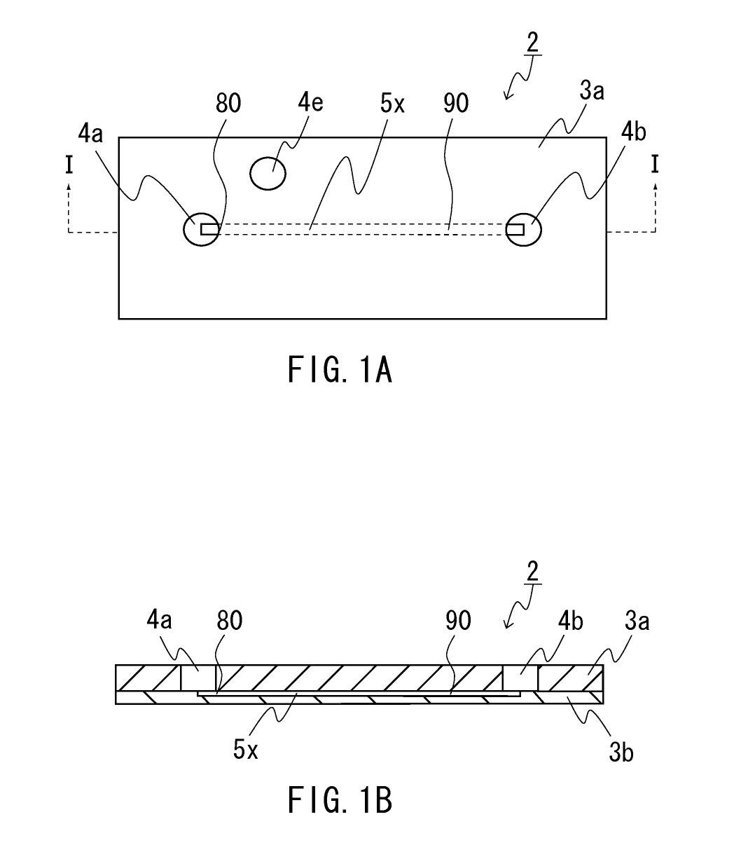

An electrophoresis chip used in the capillary electrophoresis analysis apparatus of this embodiment is shown in FIG. 1. FIG. 1 (A) shows a planar view of the electrophoresis chip. FIG. 1 (B) shows a cross-sectional view of the electrophoresis chip shown in FIG. 1 (A) viewed along the direction of line I-I. For easier understanding, the size, proportions and like features of each component in the illustrations are different from the actual features of each component. As shown in FIG. 1, the electrophoresis chip 2 of this particular embodiment is composed of a lower substrate 3b and an upper substrate 3a, the upper substrate 3a being laminated onto the lower substrate 3b. Three through-holes are formed in the upper substrate 3a. The bottom portions of the three through-holes formed in the upper substrate 3a are sealed with the lower substrate 3b, forming three fluid reservoirs 4a, 4b, and 4e. A groove having a shape of an “I” is formed on the lower substrate 3b. The upper part of the groove having a shape of an “I” formed on the lower substrate 3b is sealed with the upper substrate 3a and, forming a capillary channel 5x for sample analysis. The liquid reservoir 4a and the liquid reservoir 4b are in communication with each other via the capillary channel 5x. In contrast, the liquid reservoir 4e is not in communication with the capillary channel 5x and is provided as an independent liquid reservoir. An end of the capillary channel 5x at the liquid reservoir 4a side serves as an electrophoresis starting point 80. Further, a point on the capillary channel 5x between the liquid reservoir 4a and the liquid reservoir 4b serves as a detecting point 90. The electrophoresis chip 2 of this embodiment is rectangular parallelepiped. In the present invention, the electrophoresis chip may be in any form as long as it does not adversely affect the analysis of the sample. Furthermore, while the electrophoresis chip 2 of this embodiment includes two substrate pieces (an upper substrate 3a and a lower substrate 3b, the present invention is not limited thereto. In the present invention, the electrophoresis chip may be composed of a single-piece substrate.

In the electrophoresis chip 2 of this embodiment, the length and the width of the upper substrate 3a correspond to the maximum length and the maximum width of the whole electrophoresis chip described above. Therefore, the length and the width of the upper substrate 3a are arranged to be identical to the maximum length and the maximum width of the whole electrophoresis chip described above, respectively. The thickness of the upper substrate 3a in the electrophoresis chip 2 of this embodiment can be set suitably according to the volume of the plural liquid reservoirs 4a, 4b, and 4e—for example in the range of about 0.1 mm to about 3 mm, such as about 1 mm to about 2 mm.

In the electrophoresis chip 2 of this embodiment, the length and the width of the lower substrate 3b are the same as the length and the width of the upper substrate 3a, respectively. The thickness of the lower substrate 3b is not particularly limited, however, and in an exemplary embodiment, is in the range of about 0.1 mm to about 3 mm, such as about 0.1 mm to about 1 mm.

The material of the upper substrate 3a and the lower substrate 3b is not particularly limited as long as it does not adversely affect the measurement of the sample absorbance.

With respect to the width and the depth of the capillary channel for sample analysis 5x, the width thereof is, for example, in the range of about 25 μm to about 100 μm and the depth thereof is in the range of about 25 μm to about 100 μm. Further, the distance from the electrophoresis starting point 80 to the detecting point 90 is, for example, in the range of about 0.5 cm to about 15 cm, such as about 1 cm to about 5 cm.

The volumes of the liquid reservoirs 4a, 4b, and 4e are as described above. In FIG. 1, the liquid reservoirs 4a, 4b, and 4e are cylinders. However, the invention is not so limited. Further, the liquid reservoirs 4a, 4b, and 4e may be in any form.

In the electrophoresis chip 2 of this embodiment, the maximum thickness of the chip is the sum of the thickness of the upper substrate 3a and the lower substrate 3b. The thickness of the whole chip is as described herein.

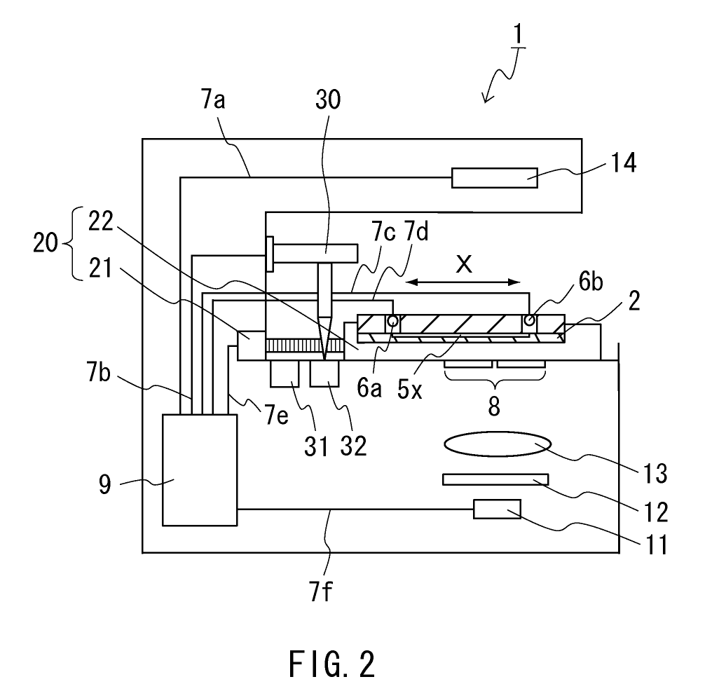

The capillary electrophoresis analysis apparatus of this embodiment is shown in FIG. 2 and includes the aforementioned electrophoresis chip 2, electrodes 6a and 6b, electric wires 7a to 7f, a slit 8, a control unit 9, a light source 11, an optical filter 12, a collecting lens 13, a detection unit 14, an electrophoresis chip transfer unit 20, a quantitative dispensing unit 30, a diluent 31, and an electrophoresis running buffer 32 as main components. The electrophoresis chip transfer unit 20 contains a drive unit 21 and a stage 22. The electrophoresis chip 2 is arranged on the stage 22. The electrodes 6a and 6b are arranged in the liquid reservoirs 4a and 4b of the electrophoresis chip 2, respectively. The detection unit 14, the quantitative dispensing unit 30, the electrodes 6a and 6b, the electrophoresis chip transfer unit 20, and the light source 11 are connected to the control unit 9 via the electric wires 7a to 7f, respectively. The control unit 9 controls power supply or the like to the aforementioned components which are connected thereto via the electric wires 7a to 7f.

In the capillary electrophoresis analysis apparatus 1 of this embodiment, the stage 22 is movable in a horizontal biaxial direction (an X-Y direction) by the drive unit 21 that is connected to an end thereof. The X direction and the Y direction vertically intersect on the horizontal surface. Thereby, the position of the electrophoresis chip 2 can be adjusted. Due to adjustment of the position of the electrophoresis chip 2, the detecting point 90 can accurately be irradiated with the light flux of specific wavelength. Further, the quantitative dispensing unit 30 can perform a quantitative analysis of the diluent 31 and the electrophoresis running buffer 32, respectively, and can dispense them to the liquid reservoir 4a or the liquid reservoir 4e of the electrophoresis chip 2. By applying the voltage between the electrodes 6a and 6b, an electrophoresis of a sample that is introduced into the capillary channel for sample analysis 5x can be performed. Then, the light emitted from the light source 11 is dispersed into specific wavelength by the optical filter 12 and converged by the collecting lens 13, as well as the amount of light is increased and the stray light is removed by the slit 8, and then the sample at the detecting point 90 on the capillary channel 5x of the electrophoresis chip 2 is irradiated. The transmitted light of the light irradiated on the detecting point 90 is detected by the detection unit 14 and an absorbance is measured. Thereby, protein in blood to be analyzed contained in the sample can be analyzed.

The method of producing the electrophoresis chip 2 of the capillary electrophoresis analysis apparatus 1 of this embodiment is not particularly limited and conventionally known methods can suitably be applied.

Next, the method of analyzing the protein in blood using the capillary electrophoresis analysis apparatus 1 of this embodiment is explained.

First, the electrophoresis running buffer 32 is prepared. The electrophoresis running buffer 32 may be the aforementioned electrophoresis running buffer but it is not particularly limited. For example, a solution prepared by adding chondroitin sulfate C in a proportion of 0.8 wt % to a solution of 100 mmol/L containing fumaric acid and arginine acid can be used, where the pH of the solution is adjusted to 4.8. Next, the electrophoresis chip 2 is attached to the stage 22 and disposed in the capillary electrophoresis analysis apparatus 1. Then, the electrophoresis running buffer 32 is injected into the liquid reservoir 4a using the quantitative dispensing unit 30. Further, the pressure in the capillary channel for sample analysis 5x is reduced by a pressure reduction pump (not shown) that is connected to the liquid reservoir 4b and the capillary channel 5x is filled with the electrophoresis running buffer 32.

Next, the diluent 31 is injected into the liquid reservoir 4e using the quantitative dispensing unit 30. Further, a human whole blood is added to the reservoir 4e as a sample and is stirred by pipetting, and thus a mixture of the sample and the diluent 31 is prepared. As the diluent 31, distilled water or the like can be used. Subsequently, the mixture is injected into the liquid reservoir 4a. Then, the voltage is applied to the electrodes 6a and 6b, which are respectively arranged in the liquid reservoirs 4a and 4b, thereby creating a potential difference between both ends of the capillary channel for sample analysis 5x. The sample is thereby moved from the electrophoresis starting point 80 to the liquid reservoir 4b side. In an exemplary embodiment, the voltage is in the range of 0.5 to 20 kV, such as about 1 kV to about 15 kV, such as about 3 kV to about 12 kV, such as about 5 kV to about 10 kV. As described above, the electric field for separation of the capillary channel for sample analysis 5x due to the voltage application can be set suitably according to the distance from the electrophoresis starting point to the detecting point and the width and the depth of the capillary channel. The electric field for separation of the capillary channel 5x is, for example, in the range of 150 V/cm to 700 V/cm, such as about 200 V/cm to about 650 V/cm, such as about 300 V/cm to about 600 V/cm, such as about 400 V/cm to about 500 V/cm.

Next, in the same manner as described above, the light is dispersed and collected, and then the detecting point 90 is irradiated with light of a wave length of 415 nm from which stray light is further removed. Then, the transmitted light at the detecting point 90 is detected by the detection unit 14 and the absorbance of the protein in blood in the sample is measured. An electropherogram is generated that indicates the relationship between the degree of the obtained absorbance and the analysis time (i.e., the time from the start of electrophoresis to detection).

### Embodiment 2

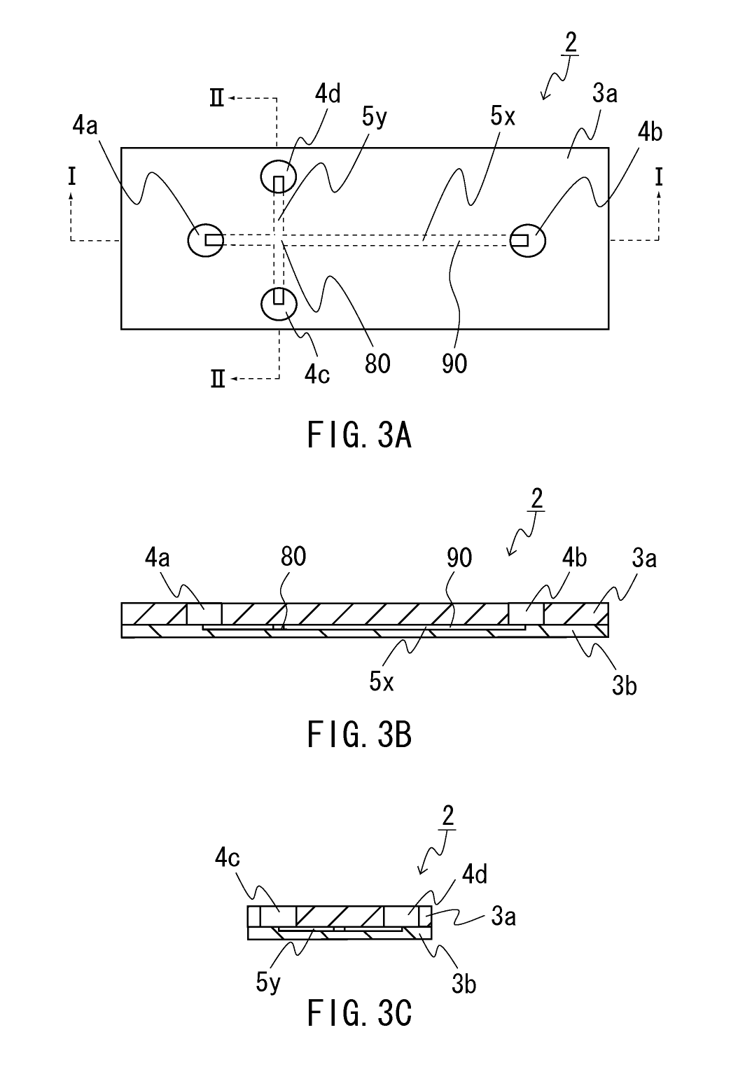

An electrophoresis chip used for the capillary electrophoresis analysis apparatus of this embodiment is shown in FIG. 3. In FIG. 3, the features that are identical to those in FIG. 1 are given the same numbers and symbols. FIG. 3 (A) shows a planar view of the electrophoresis chip of this embodiment, FIG. 3 (B) is a cross-sectional view of the electrophoresis chip shown in FIG. 3 (A) viewed along the direction of line I-I, and FIG. 3 (C) is a cross-sectional view of the electrophoresis chip shown in FIG. 3 (A) viewed along the direction of line II-II. As shown in FIG. 3, the electrophoresis chip 2 of this embodiment is composed of a lower substrate 3b and an upper substrate 3a, the upper substrate 3a being laminated onto the lower substrate 3b. Plural through-holes (four in this embodiment) are formed in the upper substrate 3a. The bottom parts of the four through-holes formed in the upper substrate 3a are sealed with the lower substrate 3b and, thereby four liquid reservoirs 4a to 4d are formed. A cross-shaped groove is formed on the lower substrate 3b. By sealing the upper part of the cross-shaped groove formed on the lower substrate 3b with the upper substrate 3a, a capillary channel for sample analysis 5x and a capillary channel for sample introduction 5y are formed. The liquid reservoir 4a and the liquid reservoir 4b are in communication with each other via the capillary channel 5x. The liquid reservoir 4c and the liquid reservoir 4d are in communication with each other via the capillary channel for sample introduction 5y. The capillary channel for sample analysis 5x and the capillary channel for sample introduction 5y intersect. The capillary channel 5x and the capillary channel 5y are in communication with each other at the intersection. The intersection serves as an electrophoresis starting point 80. Further, a point on the capillary channel 5x between the liquid reservoir 4a and the liquid reservoir 4b serves as a detecting point 90.

In the electrophoresis chip 2 of this embodiment, the maximum length of the capillary channel for sample analysis 5x is different from that of the capillary channel for sample introduction 5y. However, the present invention is not limited thereto. In the present invention, the maximum length of the capillary channel 5x of the electrophoresis chip may be the same as the maximum length of the capillary channel 5y of the electrophoresis chip.

The electrophoresis chip 2 of this embodiment has the same configuration as the electrophoresis chip shown in FIG. 1 except that the liquid reservoirs 4c and 4d and the capillary channel for sample introduction 5y are formed as well as the liquid reservoir 4e is not formed. The width and the depth of the capillary channel for sample introduction 5y are the same as the width and the depth of the capillary channel for sample analysis 5x. The distance from the electrophoresis starting point 80 to the detecting point 90 is the same as that of the electrophoresis chip shown in FIG. 1. The volume and the form of the liquid reservoirs 4c and 4d are the same as that of the electrophoresis chip shown in FIG. 1.

A capillary electrophoresis analysis apparatus of this embodiment has the same configuration as the capillary electrophoresis analysis apparatus shown in FIG. 2 except that the electrophoresis chip 2 is the electrophoresis chip shown in FIG. 3 instead of the electrophoresis chip shown in FIG. 1 as well as electrodes 6c and 6d (not shown) are arranged in the liquid reservoirs 4c and 4d of the electrophoresis chip 2.

Next, the method of analyzing the protein in blood using the capillary electrophoresis analysis apparatus 1 of this embodiment is explained.

First, the electrophoresis chip 2 is attached to a stage 22 and disposed in the capillary electrophoresis analysis apparatus 1 of this embodiment. Subsequently, in the same manner as in Embodiment 1, the electrophoresis running buffer 32 is injected into the liquid reservoir 4a using the quantitative dispensing unit 30. Next, in the same manner as in Embodiment 1, the pressure in the capillary channel for sample analysis 5x is reduced by a pressure reduction pump (not shown) that is connected to the liquid reservoir 4b, and the capillary channel 5x is filled with the electrophoresis running buffer 32. Then, the electrophoresis running buffer 32 is injected into the liquid reservoir 4c using the quantitative dispensing unit 30. Further, the pressure in the capillary channel for sample introduction 5y is reduced by a pressure reduction pump (not shown) that is connected to the liquid reservoir 4d, and the capillary channel 5y is filled with the electrophoresis running buffer 32.

Next, the diluent 31 is injected into the liquid reservoir 4c using the quantitative dispensing unit 30. Further, a human whole blood is added thereto as a sample and is stirred by pipetting. Then, the voltage is applied to the electrodes 6c and 6d, thereby creating a potential difference between both ends of the capillary channel for sample introduction 5y. The sample is thereby moved to the intersection of the capillary channel for sample analysis 5x and the capillary channel for sample introduction 5y. The voltage applied between the electrodes 6c and 6d is not particularly limited, and is, for example, in the range of about 0.5 to about 20 kV, such as about 1 kV to about 15 kV, such as about 3 kV to about 12 kV, such as about 5 kV to about 10 kV.

Next, the voltage is applied to the electrodes 6a and 6b, thereby creating a potential difference between both ends of the capillary channel for sample analysis 5x. The sample is thereby moved from the electrophoresis starting point 80 to the liquid reservoir 4b side. The voltage is not particularly limited, however is, for example, in the range of about 0.5 to about 20 kV. As described above, the electric field for separation of the capillary channel 5x due to the voltage application can be set suitably according to the distance from the electrophoresis starting point to the detecting point and the width and the depth of the capillary channel. However, the electric field for separation of the capillary channel 5x is, for example, in the same range as Embodiment 1.

Next, in the same manner as in Embodiment 1, the light is dispersed and collected, and then the detecting point 90 is irradiated with light at a wave length of 415 nm, from which a stray light is further removed. Then, the transmitted light at the detecting point 90 is detected using the detection unit 14 and the absorbance of the protein in the sample is measured. An electropherogram is generated that indicates the relationship between the degree of the obtained absorbance and the electrophoresis time.

### Embodiment 3

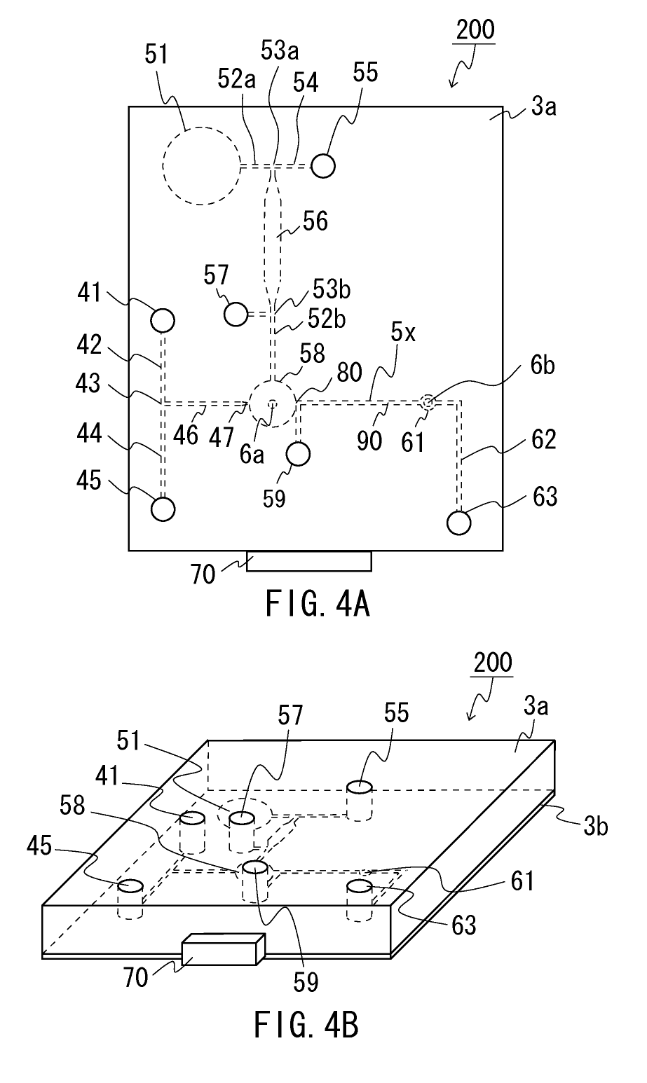

An electrophoresis chip used for the capillary electrophoresis analysis apparatus of this embodiment is shown in FIG. 4. In FIG. 4, the portions that are identical to those in FIG. 1 and FIG. 3 are given the same numbers and symbols. FIG. 4 (A) shows a planar view of the electrophoresis chip of this embodiment, and FIG. 4 (B) is a perspective view of the electrophoresis chip of this embodiment. As shown in FIG. 4, the electrophoresis chip 2 of this embodiment includes a laminated body, in which an upper substrate 3a is laminated onto a lower substrate 3b, and a connector 70. The connector 70 is arranged on a side surface of the laminated body. A wiring pattern (not shown) is formed on the lower substrate 3b. Six through-holes are formed in the upper substrate 3a. The bottom parts of the six through-holes are sealed with the lower substrate 3b, and thereby six liquid reservoirs are formed. The six liquid reservoirs serve as a sample introduction portion 41, a drain 45, a drain 55, a drain 57, a drain 59, and a drain 63, respectively. Further, three concave portions of various sizes are formed at the bottom surface of the upper substrate 3a. Openings of two concave portions out of the three concave portions are sealed with the lower substrate 3b, and thereby two liquid reservoirs are formed. The two liquid reservoirs serve as a reagent reservoir 51 and a diluent reservoir 58, respectively. An electrophoresis running buffer is sealed in the reagent reservoir 51. An electrode 6a connected to a wiring of the wiring pattern is arranged in the diluent reservoir 58, and a stirring bar (not shown) is sealed in the diluent reservoir 58. An opening of the other one of the concave portion out of the three concave portions is sealed with the lower substrate 3b, and thereby an electrode arrangement portion 61 is formed. An electrode 6b connected to a wiring of the wiring pattern is arranged in the electrode arrangement portion 61. Further, plural grooves are formed on the bottom surface of the upper substrate 3a. Openings of the plural grooves are sealed with the lower substrate 3b, and thereby channels are formed, through which the six reservoirs and the three concave portions are in communication with one another. A capillary channel, through which the diluent reservoir 58 and the electrode arrangement portion 61 are in communication with each other, serves as the capillary channel for sample analysis 5x. An end portion of the capillary channel 5x at the diluent reservoir 58 side serves as an electrophoresis starting point 80. Further, a point on the capillary channel 5x serves as a detecting point 90. Details of channels other than the capillary channel 5x are described herein.

The sample introduction portion 41 is in communication with the drain 45 via a sample introduction channel 42, a branching portion 43, and an overflow channel 44 in order. Further, the sample introduction portion 41 is also in communication with the diluent reservoir 58 from the branching portion 43 via a sample measurement channel 46. The sample introduction portion 41 is an introduction opening for introducing a sample, which contains protein in blood to be analyzed, into an electrophoresis chip. At an end portion of the sample measurement channel 46 at the diluent reservoir 58 side, an orifice 47 having a narrow channel cross-sectional area is formed.

In the electrophoresis chip 2 of this embodiment, the sample is measured and introduced into the electrophoresis chip in the following manner. First, after introducing a sample into the sample introduction portion 41, the sample is suctioned from the drain 45 with a pressure reduction pump (not shown). Due to the suction, a sample that exceeds the volume of the sample measurement channel 46 between the branching portion 43 and the orifice 47 flows into the overflow channel 44. Subsequently, the drain 45 is closed and an air is discharged from the sample introduction portion 41 with a pressure pump (not shown) or the like. Thereby, a sample corresponding to the volume of the sample measurement channel 46 stored therein is measured and introduced into the diluent reservoir 58.

The reagent reservoir 51 is in communication with the drain 55 via a reagent introduction channel 52a, a branching portion 53a, and an overflow channel 54 in order. Further, the reagent reservoir 51 is also in communication with the diluent reservoir 58 from the branching portion 53a via a reagent measurement channel 56, a branching portion 53b, and a reagent introduction channel 52b. At an end portion of a channel that is branched at the branching portion 53b, the drain 57 is formed. Further, at an end portion of a channel that is branched at an end portion of the capillary channel for sample analysis 5x at the diluent reservoir 58 side, a drain 59 is formed. Furthermore, between the electrode arrangement portion 61 and the drain 63, a flow amount measurement channel 62 is formed.

In the electrophoresis chip 2 of this embodiment, the capillary channel for sample analysis 5x is filled with the electrophoresis running buffer, and the electrophoresis running buffer is measured and introduced into the diluent reservoir 58 in the following manner. First, the sample introduction portion 41, the drains 45, 55, 57, and 59 are closed, and air is suctioned with a pressure reduction pump (not shown) or the like that is connected to the drain 63. Thereby, the reagent introduction channels 52a and 52b, the reagent measurement channel 56, the diluent reservoir 58, the capillary channel for sample analysis 5x, the electrode arrangement portion 61, and the flow amount measurement channel 62 are filled with an electrophoresis running buffer which is sealed in the reagent reservoir 51. Subsequently, the reagent reservoir 51 is closed, the drain 59 is opened, and air is suctioned with a pressure reduction pump (not shown) or the like that is connected to the drain 57. Thereby, an electrophoresis running buffer in the reagent introduction channel 52b and the diluent reservoir 58 is removed. Further, the drain 57 is closed, the drain 55 is opened, and air is suctioned with a pressure reduction pump (not shown) or the like that is connected to the drain 59. Thereby, an electrophoresis running buffer corresponding to the volume of the reagent measurement channel 56 can be measured and introduced into the diluent reservoir 58. Further, the sample and the electrophoresis running buffer can be mixed by introducing the sample into the diluent reservoir 58 as described above and rotating the stirring bar (not shown) using a magnetic stirrer (not shown).

In the electrophoresis chip 2 of this embodiment, the material comprising the upper substrate 3a is not particularly limited as long as it does not adversely affect the measurement of the absorbance. For example, the upper substrate 3a formed of the aforementioned materials can be used.

In the electrophoresis chip 2 of this embodiment, the length and the width of the upper substrate 3a are, for example, in the range of about 10 mm to about 200 mm, such as about 20 mm to about 100 mm. Further, the thickness of the upper substrate 3a is, for example, in the range of about 0.1 mm to about 10 mm, such as in the range of about 1 mm to about 5 mm.

In the electrophoresis chip 2 of this embodiment, the lower substrate 3b, which is formed of acrylic resin, the material similar to that of the upper substrate 3a, or the like, can be used. The lower substrate 3b is prepared by laminating plural substrates formed of the aforementioned materials. Between the plural substrates, wiring patterns made of copper foil or the like are formed.

In this embodiment, the length and the width of the lower substrate 3b are the same as that of the upper substrate 3a. The thickness of the lower substrate 3b is, for example, in the range of about 0.1 mm to about 10 mm.

In the electrophoresis chip 2 of this embodiment, with respect to the diameter and the depth of the sample introduction portion 41, the diameter is in the range of about 0.1 mm to about 10 mm, such as about 1 mm to about 5 mm, and the depth is in the range of about 0.1 mm to about 10 mm, such as about 1 mm to about 5 mm.

In the electrophoresis chip 2 of this embodiment, with respect to the diameter and the depth of the reagent reservoir 51, the diameter is in the range of about 0.5 mm to about 50 mm, such as about 1 mm to about 20 mm, and the depth is in the range of about 0.1 mm to about 10 mm, such as about 1 mm to about 5 mm. Further, in the electrophoresis chip of this embodiment, with respect to the diameter and the depth of the diluent reservoir 58, the diameter is in the range of about 0.5 mm to about 50 mm, such as about 1 mm to about 10 mm, and the depth is in the range of about 0.1 mm to about 10 mm, such as about 1 mm to about 5 mm.

In the electrophoresis chip 2 of this embodiment, with respect to the diameter and the depth of the drains 45, 55, 57, 59, and 63, the diameter is in the range of about 0.1 mm to about 10 mm, such as about 1 mm to about 5 mm, and the depth is in the range of about 0.1 mm to about 10 mm, such as about 1 mm to about 5 mm.

In the electrophoresis chip 2 of this embodiment, the form of the sample introduction portion 41, the reagent reservoir 51, the diluent reservoir 58, and the drains 45, 55, 57, 59, and 63 is cylindrical. However, the present invention is not so limited. In the invention, the sample introduction portion 41, the reagent reservoir 51, the diluent reservoir 58, and the drains 45, 55, 57, 59, and 63 may be in an arbitrary form, with examples thereof including a quadrangular prism, a quadrangular pyramid, a cone, a combination thereof. The form of the sample introduction portion 41, the reagent reservoir 51, the diluent reservoir 58, and the drains 45, 55, 57, 59, and 63 may all be the same or may each be different.

In the electrophoresis chip 2 of this embodiment, the width and the depth of the capillary channel for sample analysis 5x are the same as that of the electrophoresis chip shown in FIG. 1. Further, the distance from the electrophoresis starting point 80 to the detecting point 90 is the same as that of the electrophoresis chip shown in FIG. 1.

In the electrophoresis chip 2 of this embodiment, with respect to the width and the depth of the reagent measurement channel 56 at the maximum portion of a cross-sectional area, the width is in the range of about 0.1 mm to about 10 mm and the depth is in the range of about 0.1 mm to about 10 mm.

In the electrophoresis chip 2 of this embodiment, with respect to the width and the depth of the orifice 47, the width is in the range of about 1 μm to about 200 μm, such as about 10 μm to about 100 μm, and the depth is in the range of about 1 μm to about 200 μm, such as about 10 μm to about 100 μm.

In the electrophoresis chip 2 of this embodiment, with respect to the width and the depth of capillary channels except for the capillary channel for sample analysis 5x, the reagent measurement channel 56, and the orifice 47, the width is in the range of about 10 μm to about 1000 μm, such as about 50 μm to about 500 μm, and the depth is in the range of about 10 μm to about 1000 μm, such as about 50 μm to about 500 μm.

In the electrophoresis chip 2 of this embodiment, the maximum thickness of the whole electrophoresis chip is a sum of the thickness of the upper substrate 3a and the lower substrate 3b. The thickness of the whole electrophoresis chip is as described above.

The method of producing the electrophoresis chip 2 of this embodiment is not particularly limited and conventionally known methods can suitably be used.

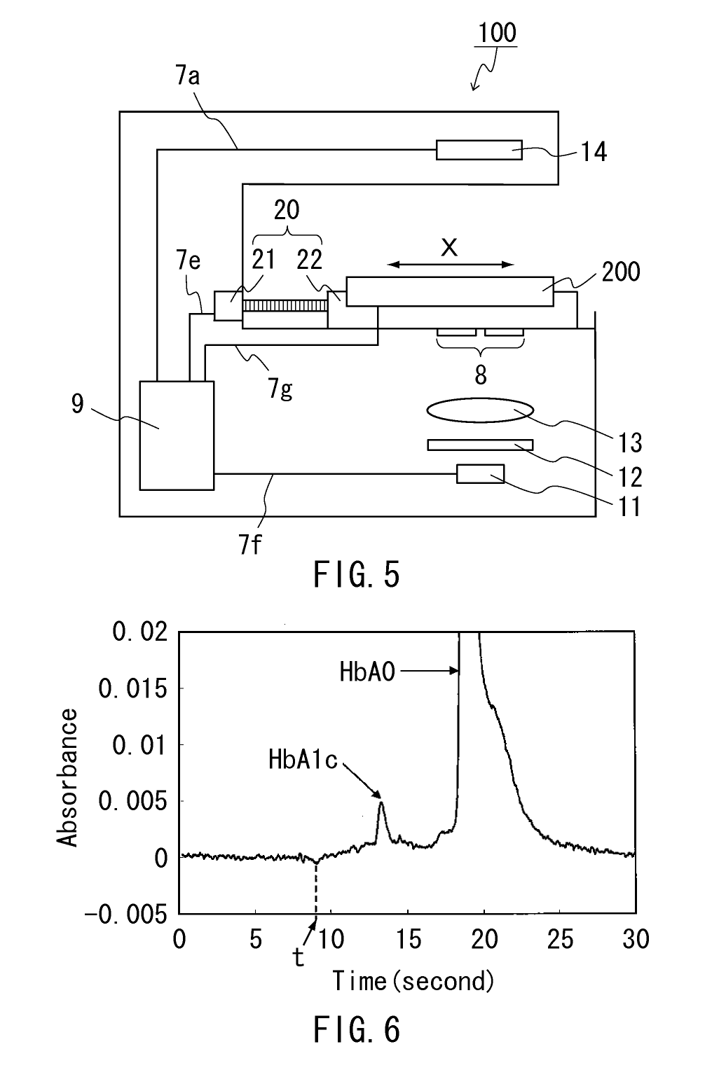

A capillary electrophoresis analysis apparatus of this embodiment is shown in FIG. 5, wherein the features that are identical to those in FIG. 2 are given the same numbers and symbols. As shown in FIG. 5, the capillary electrophoresis analysis apparatus 1 has the same configuration as the electrophoresis analysis apparatus shown in FIG. 2 except that an electrophoresis chip 2 is the electrophoresis chip shown in FIG. 4 instead of the electrophoresis chip shown in FIG. 1, the quantitative dispensing unit 30, the diluent 31, and the electric wires 7b to 7d are not provided as well as the electrophoresis running buffer is provided in the electrophoresis chip 2 and the connecting portion (not shown) of the connector 70 and an electric wire 7g are provided. Although it is not shown in FIG. 5, the electrophoresis chip 2 is attached to the stage 22 via the connector 70 and disposed in the capillary electrophoresis analysis apparatus 1. Further, the connector 70 is connected to the control unit 9 via the electric wire 7g. The control unit 9 controls power supply or the like to the connector 70.

A method of analyzing protein in blood using the capillary electrophoresis analysis apparatus 1 of this embodiment is described as follows.

First, the electrophoresis chip 2 is attached to the capillary electrophoresis analysis apparatus 1 via the connector 70. Next, as described above, the capillary channel for sample analysis 5x is filled with the electrophoresis running buffer. Further, the electrophoresis running buffer is measured and introduced into the diluent reservoir 58. Then, in the same manner as described above, a human whole blood is introduced from the sample introduction portion 41 as the sample, and a human whole blood corresponding to the volume of the sample measurement channel 46 is measured and introduced into the diluent reservoir 58. The sample and the electrophoresis running buffer thus introduced are mixed in the diluent reservoir 58 and stirred by rotating the stirring bar (not shown) by a magnetic stirrer (not shown).

Next, the voltage is applied to the electrodes 6a and 6b, thereby creating a potential difference between both ends of the capillary channel for sample analysis 5x. The sample is thereby moved from the electrophoresis starting point 80 to the electrodes 6b side. The voltage application is performed by supplying power from the connector 70 to the electrodes 6a and 6b via the electric wire 7g. The voltage is not particularly limited, however is, for example, in the range of 0.5 to 20 kV. As described above, the electric field for separation of the capillary channel 5x due to the voltage application can be set suitably according to the distance from the electrophoresis starting point to the detecting point and the width and the depth of the capillary channel. However, the electric field for separation of the capillary channel 5x is, for example, in the same range as Embodiment 1.

Next, in the same manner as in Embodiment 1, the light is dispersed and collected, and then the detecting point 90 is irradiated with light of a wave length of 415 nm, from which stray light is further removed. Then, the transmitted light at the detecting point 90 is detected using the detection unit 14 and the absorbance of the protein in the sample that is subjected to the electrophoresis is measured. An electropherogram is generated that indicates the relationship between the degree of the obtained absorbance and the analysis time.

### EXAMPLES

The following examples describe the analysis of samples containing glycosylated hemoglobin (HbA1c) using the capillary electrophoresis analysis apparatus of the present invention shown in FIG. 2 are explained.

### Example 1

An electrophoresis chip as shown in FIG. 1 was produced from polymethylmethacrylate (PMMA). The length (dimension in the direction along the capillary channel for sample analysis 5x) of the chip 2 was about 70 mm and the width thereof was about 30 mm. The width of the capillary channel for sample analysis 5x was about 40 μm and the depth thereof was about 40 μm. Further, the distance from the electrophoresis starting point 80 to the detecting point 90 was about 1.5 cm.

The electrophoresis chip 2 was then attached to the stage 22 and the capillary electrophoresis analysis apparatus 1 shown in FIG. 2 was configured. The electrophoresis running buffer 32 was prepared by adding chondroitin sulfate C in a proportion of 0.8 wt % to a solution, in which 30 mmol/L of sodium thiocyanate was added to 50 mmo/L of fumaric acid that had been adjusted to pH 4.8 by the addition of arginine. Using the electrophoresis running buffer 32, hemoglobin (manufactured by BML INC.) was diluted to a concentration of 10 g/L, and thereby the sample to be electrophoresed was prepared. In this state, the hemoglobin A1c concentration was about 5 wt % and the hemoglobin concentration thereof was corresponding to a hemoglobin concentration of a human blood that is diluted by 15 fold.

The electrophoresis running buffer 32 was then injected into the liquid reservoir 4a of the electrophoresis chip 2. The capillary channel for sample analysis 5x was filled with the electrophoresis running buffer 32 by suction with a pressure reduction pump that was connected to the liquid reservoir 4b. The sample was then introduced into the liquid reservoir 4a. An electric field for separation of 450 V/cm was then applied to the electrodes 6a and 6b, thereby creating a potential difference between both ends of the capillary channel 5x. Thereby, the sample was moved from the liquid reservoir 4a to the liquid reservoir 4b side. At that time, an absorbance (with a measurement wave length of 415 nm) at the detecting point 90 of the capillary channel for sample analysis 5x was measured using the detection unit 14. Then, electropherogram was generated that indicated the relationship between the absorbance and the time elapsed from the start of the voltage application, i.e., analysis time (sec). Further, in the electropherogram, an electroosmotic flow was calculated using the following Formula (I) with a decreasing point of absorbance appeared before detection of hemoglobin being considered as t (sec) and the distance from the electrophoresis starting point to the detecting point being considered as d (cm). Electroosmotic flow (ch/min)=d/(t/60).

### Example 2

In Example 2, the electrophoresis chip 2 was prepared and the capillary electrophoresis analysis apparatus 1 was configured in the same manner as in Example 1. Using the capillary electrophoresis analysis apparatus 1, stable hemoglobin A1c and unstable hemoglobin A1c were analyzed. The analysis was performed in the same manner as in Example 1 except that 100 g/L of hemoglobin, to which 500 mg/dL of glucose had been added, and which was incubated at 37° C. and diluted 10 fold, was used as the sample instead of the 10 g/L of hemoglobin described in Example 1.

### Example 3

In Example 3, the electrophoresis chip 2 was prepared and the capillary electrophoresis analysis apparatus 1 was configured in the same manner as in Example 1. Using the capillary electrophoresis analysis apparatus 1, hemoglobin A1c was analyzed. The analysis was performed in the same manner as in Example 1 except that the length from the electrophoresis starting point 80 to the detecting point 90 was 1.0 cm instead of the 1.5 cm described in Example 1.

### Example 4

In Example 4, the electrophoresis chip 2 was prepared and the capillary electrophoresis analysis apparatus 1 was configured in the same manner as in Example 1. Using the capillary electrophoresis analysis apparatus 1, hemoglobin A1c was analyzed. The analysis was performed in the same manner as in Example 1 except that the length from the electrophoresis starting point 80 to the detecting point 90 was 2.0 cm instead of the 1.5 cm described in Example 1.

### Example 5

In Example 5, the electrophoresis chip 2 was produced and the capillary electrophoresis analysis apparatus 1 was configured in the same manner as in Example 1. Using the capillary electrophoresis analysis apparatus 1, hemoglobin A1c was analyzed. The analysis was performed in the same manner as in Example 1 except that the length from the electrophoresis starting point 80 to the detecting point 90 was 0.5 cm instead of the 1.5 cm described in Example 1, and an electric field for separation applied to the electrodes 6a and 6b was 250 V/cm instead of the 450 V/cm described in Example 1.

### Comparative Example 1

In this Example, the electrophoresis chip 2 was produced and the capillary electrophoresis analysis apparatus 1 was configured in the same manner as in Example 1. Using the capillary electrophoresis analysis apparatus 1, hemoglobin A1c was analyzed. The analysis was performed in the same manner as in Example 1 except that the hemoglobin concentration of the sample was 5 g/L (hemoglobin A1c concentration of about 11%) instead of 10 g/L, the length from the electrophoresis starting point 80 to the detecting point 90 was 2.0 cm instead of the 1.5 cm, in Example 1 and an electric field for separation applied to the electrodes 6a and 6b was 250 V/cm instead of the 450 V/cm in Example 1. The hemoglobin concentration of the sample was 5 g/L and corresponded to the hemoglobin concentration of a human blood that is diluted by 30 fold.

### Comparative Example 2

In this Example, the electrophoresis chip 2 was produced and the capillary electrophoresis analysis apparatus 1 was configured in the same manner as in Example 1 except that a separated member of capillary tube (with an inner diameter of 40 μm) that was embedded in a groove formed on the lower substrate 3b was used as the capillary channel for sample analysis 5x. The material of the capillary tube was fused silica. Using the capillary electrophoresis analysis apparatus 1, hemoglobin A1c was analyzed. The analysis was performed in the same manner as in Comparative Example 1 except that the aforementioned electrophoresis chip 2 was used.

Detection results of Examples 1 to 5 and Comparative Examples 1 and 2 are shown in the graphs of FIGS. 6 to 12. FIG. 6 shows the result of Example 1.

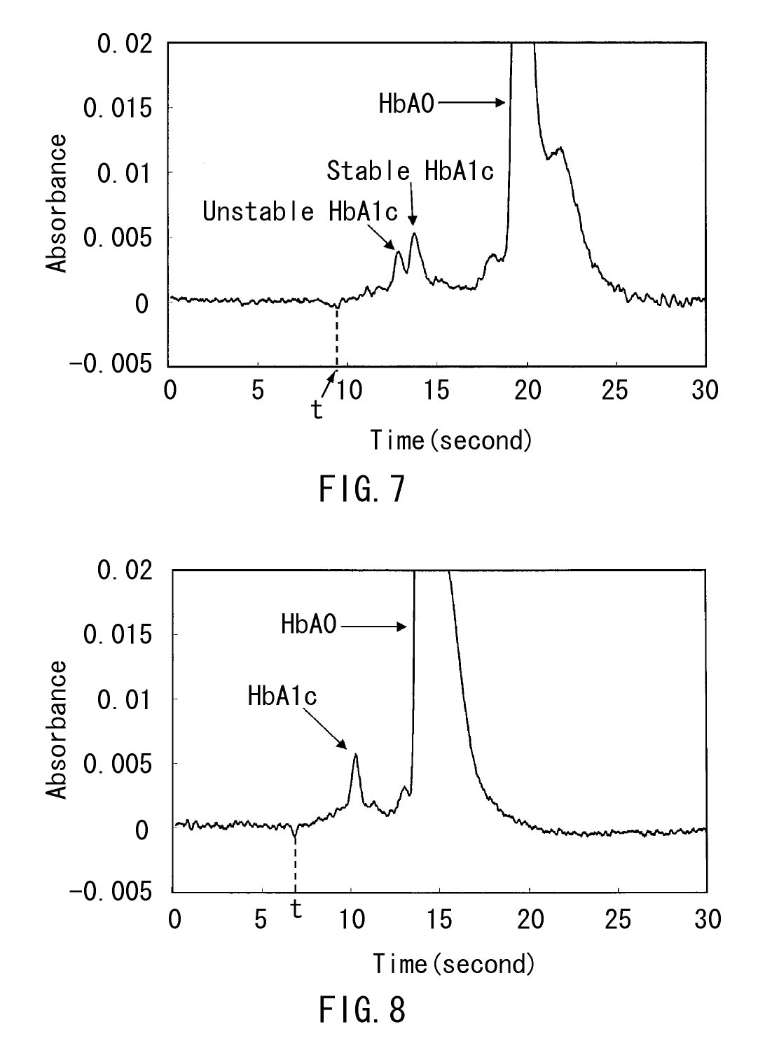

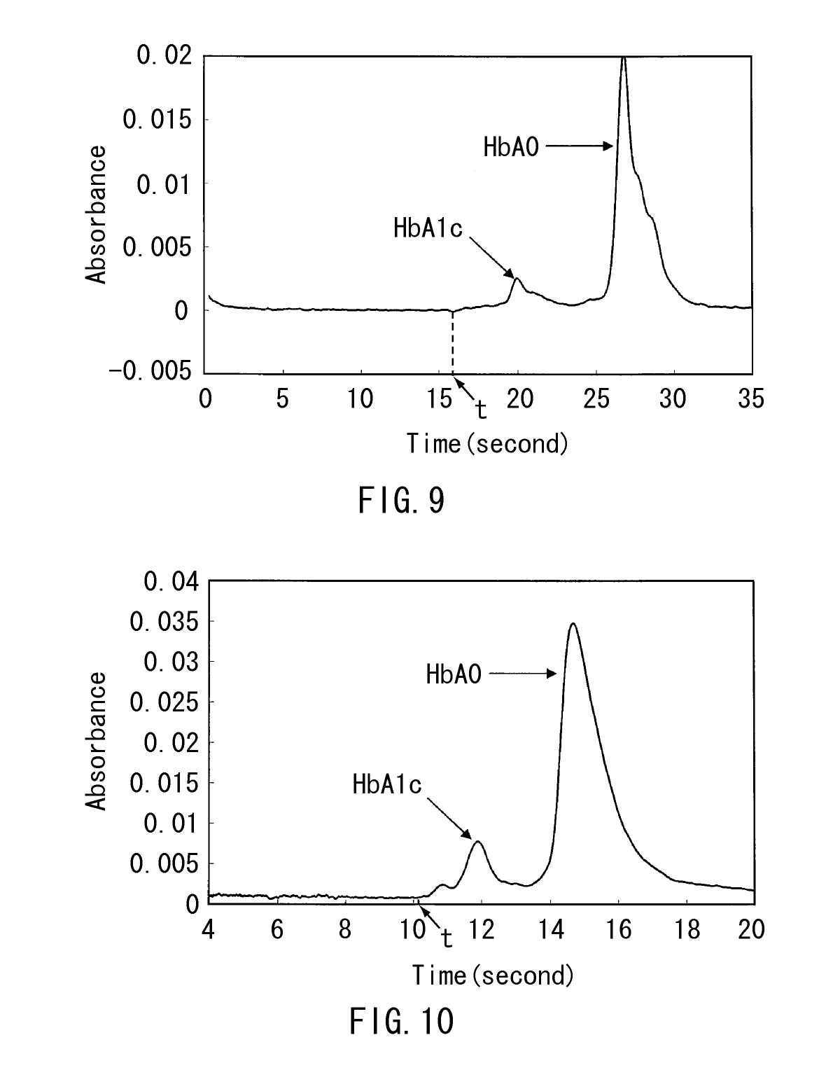

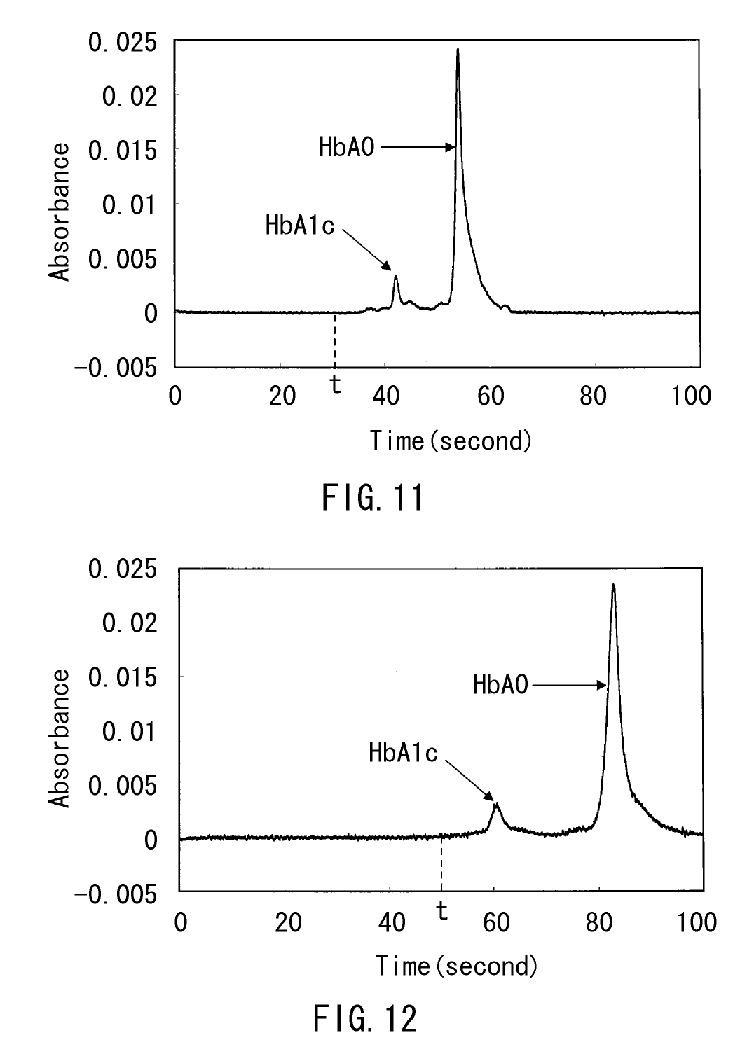

FIG. 7 shows the result of Example 2. FIG. 8 shows the result of Example 3. FIG. 9 shows the result of Example 4. FIG. 10 shows the result of Example 5. FIG. 11 shows the result of Comparative Example 1. FIG. 12 shows the result of Comparative Example 2. Further, in the graphs of FIGS. 6 to 12, horizontal axes indicate the time (sec) elapsed from the start of voltage application and vertical axes indicate an absorbance at a measurement wave length of 415 nm.

Results of electroosmotic flow calculated in Examples 1 to 5 and Comparative Examples 1 and 2 are shown in the following Table 1. As shown in Table 1, with respect to the electroosmotic flow, Examples 1 and 2 were 10 cm/min. Example 3 was 8.6 cm/min. Example 4 was 7.5 cm/min. Example 5 was 3.0 cm/min. Comparative Example 1 was 4.0 cm/min. Comparative Example 2 was 2.4 cm/min.

TABLE 1

| | t (sec) | electroosmotic flow (cm/min) |
|---|---|---|
| Example 1 | 9 | 10 |
| Example 2 | 9 | 10 |
| Example 3 | 7 | 8.6 |
| Example 4 | 16 | 7.5 |
| Example 5 | 10 | 3.0 |
| Comparative Example 1 | 30 | 4.0 |
| Comparative Example 2 | 50 | 2.4 |

As shown in graphs of FIGS. 6, and 8 to 12, in Examples 1, 3, 4, and 5, and Comparative Examples 1 and 2, hemoglobin A1c and hemoglobin A0 could be detected separately. Further, as shown in the graph in FIG. 7, in Example 2, three hemoglobins, such as hemoglobin A1c, unstable hemoglobin A1c, and hemoglobin A0, could be detected separately. The time (analysis time of hemoglobin) from the start of the voltage application to the completion of detection in each Example was about 24 seconds in Example 1, about 25 seconds in Example 2, about 18 seconds in Example 3, about 30 seconds in Example 4, and about 18 seconds in Example 5. In contrast, the time from the start of voltage application to the completion of detection was about 60 seconds in Comparative Example 1 and about 90 seconds in Comparative Example 2. Stated differently, although the analysis time in Examples 1 to 5 were very short, such as 30 seconds or shorter, the analysis time in Comparative Examples 1 and 2 were 60 seconds or longer.

According to the present invention, the whole analysis apparatus can be downsized, operation can be simplified, running cost can be reduced, and protein in blood can be analyzed highly accurately in a short time such as 30 seconds or shorter. The present invention is particularly suitable for a micro total analysis systems (μTAS) and is applicable to all technical fields where samples, such as blood proteins, are analyzed, such as laboratory tests, biochemical examinations and medical research. The intended use of the capillary electrophoresis analysis apparatus is not limited and it is applicable to a broad range of technical fields.

It should be understood that the foregoing discussions and examples merely present a detailed description of certain exemplary embodiments. It should therefore be apparent to those of ordinary skill in the art that modifications and equivalents can be made without departing from the spirit and scope of the invention. All journal articles, other references, patents and patent applications that are identified in this application are incorporated by reference in their entireties.

What is claimed is:

1. A method of analyzing a sample comprising
applying a sample to a capillary electrophoresis analysis apparatus; and
performing electrophoretic separation and detection of the sample in greater than 0 seconds but less than 35 seconds,
wherein the capillary electrophoresis analysis apparatus comprises
an electrophoresis chip comprising
a substrate;
a capillary channel; and
a plurality of liquid reservoirs in communication with each other via the capillary channel;
a voltage application unit comprising an electrode in communication with the capillary channel; and
an absorbance measurement unit, and
wherein the detection is measured by the absorbance measurement unit.

2. The method according to claim 1, wherein the apparatus has a width of about 10 cm to about 100 cm, a depth of about 10 cm to about 100 cm and a height of about 5 cm to about 100 cm.

3. The method according to claim 1, wherein the capillary channel is formed on the surface of the substrate or is a tube embedded in the substrate.

4. The method according to claim 1, wherein an inner wall surface of the capillary channel is coated with a cationic layer, an anionic layer or a neutral layer.

5. The method according to claim 1, wherein the plurality of liquid reservoirs are depressions formed on the surface of the substrate.

6. The method according to claim 1, wherein the electrophoresis chip has a length of about 10 mm to about 100 mm, a width of about 10 mm to about 60 mm and a thickness of about 0.3 mm to about 5 mm.

7. The method according to claim 1, wherein the capillary channel contains an electrophoresis running buffer, wherein the electrophoresis running buffer comprises a sulfated polysaccharide.

8. The method according to claim 7, wherein the sulfated polysaccharide is a chondroitin sulfate.

9. The method according to claim 1, wherein the capillary channel has a diameter of about 25 μm to about 100 μm and a length of about 0.5 cm to about 15 cm.

10. The method according to claim 1, wherein the capillary channel contains a cross-sectional shape perpendicular to the channel direction.

11. The method according to claim 10, wherein the cross-sectional shape is circular, rectangular, ellipsoidal or polygonal.

12. The method according to claim 11, wherein when the cross-sectional shape is circular, the diameter thereof is about 25 μm to about 100 μm.

13. The method according to claim 11, wherein when the cross-sectional shape is rectangular, the width thereof is about 25 μm to about 100 μm and the depth thereof is about 25 μm to about 100 μm.

14. The method according to claim 1, wherein the capillary electrophoresis apparatus further comprises a pre-filter component, an air vent structure, a stray light removing unit, a position adjustment unit, a quantitative dispensing unit, a stirring unit, a liquid sending unit or combinations thereof.

15. The method according to claim 1, wherein the electrophoresis chip surface has been treated with at least one of phosphoric acid, UV radiation, alkali dipping, an inorganic nanomicroparticle coating, graft co-polymerization and corona discharge to minimize adsorption of the sample.

16. The method according to claim 7, wherein the electrophoresis running buffer further comprises a chaotropic anion.

17. The method according to claim 1, wherein the sample comprises a blood protein.

18. The method according to claim 17, wherein the blood protein comprises hemoglobin.

19. The method according to claim 18, wherein the hemoglobin is at least one of normal hemoglobin, glycosylated hemoglobin, modified hemoglobin, variant hemoglobin, and fetal hemoglobin.

20. The method according to claim 18, wherein the hemoglobin is at least one of hemoglobin A1c, hemoglobin F, hemoglobin A2, hemoglobin S, and hemoglobin C.

21. The method according to claim 20, wherein the hemoglobin is hemoglobin A1c.

22. The method according to claim 1, wherein the sample comprises hemoglobin and wherein a concentration of the hemoglobin is detected by the absorbance measurement unit.

23. The method according to claim 22, wherein the absorbance measurement unit measures absorbance by the hemoglobin at a wavelength range of about 260 nm to about 300 nm or at a range of about 380 nm to about 450 nm.

24. The method according to claim 1, wherein the sample comprises hemoglobin and is subjected to a hemolysis treatment.

25. The method according to claim 24, wherein the hemolysis treatment is at least one of a surfactant treatment, an osmotic pressure treatment, and a sonication treatment.

26. The method according to claim 1, wherein an electroosmotic flow generated during electrophoretic separation of the sample is in the range of about 3 to about 20 cm/min.

27. A method of diagnosing diabetes in a subject comprising
obtaining a sample of blood from a subject;
applying the sample to a capillary electrophoresis apparatus; and
performing electrophoretic separation and detection of the sample for determining the amount of glycated hemoglobin in the sample, thereby determining whether the subject has diabetes,
wherein the capillary electrophoresis analysis apparatus comprises
an electrophoresis chip comprising
a substrate;
a capillary channel; and
a plurality of liquid reservoirs in communication with each other via the capillary channel;
a voltage application unit comprising an electrode in communication with the capillary channel; and
an absorbance measurement unit, and
wherein the detection is measured by the absorbance measurement unit.

28. The method according to claim 27, wherein the apparatus has a width of about 10 cm to about 100 cm, a depth of about 10 cm to about 100 cm and a height of about 5 cm to about 100 cm.

29. The method according to claim 27, wherein the capillary channel is formed on the surface of the substrate or is a tube embedded in the substrate.

30. The method according to claim 27, wherein an inner wall surface of the capillary channel is coated with a cationic layer, an anionic layer or a neutral layer.

31. The method according to claim 27, wherein the plurality of liquid reservoirs are depressions formed on the surface of the substrate.

32. The method according to claim 27, wherein the electrophoresis chip has a length of about 10 mm to about 100 mm, a width of about 10 mm to about 60 mm and a thickness of about 0.3 mm to about 5 mm.

33. The method according to claim 27, wherein the capillary channel contains an electrophoresis running buffer, wherein the electrophoresis running buffer comprises a sulfated polysaccharide.

34. The method according to claim 33, wherein the sulfated polysaccharide is a chondroitin sulfate.

35. The method according to claim 27, wherein the capillary channel has a diameter of about 25 μm to about 100 μm and a length of about 0.5 cm to about 15 cm.

36. The method according to claim 27, wherein the capillary channel contains a cross-sectional shape perpendicular to the channel direction.

37. The method according to claim 36, wherein the cross-sectional shape is circular, rectangular, ellipsoidal or polygonal.

38. The method according to claim 37, wherein when the cross-sectional shape is circular, the diameter thereof is about 25 μm to about 100 μm.

39. The method according to claim 37, wherein when the cross-sectional shape is rectangular, the width thereof is about 25 μm to about 100 μm and the depth thereof is about 25 μm to about 100 μm.

40. The method according to claim 27, wherein the capillary electrophoresis apparatus further comprises a pre-filter component, an air vent structure, a stray light removing unit, a position adjustment unit, a quantitative dispensing unit, a stirring unit, a liquid sending unit or combinations thereof.

41. The method according to claim 33, wherein the electrophoresis running buffer further comprises a chaotropic anion.

42. The method according to claim 27, wherein the electrophoretic separation and detection of the sample occurs in greater than 0 seconds but less than 35 seconds.

43. A method of monitoring diabetes in a subject comprising
obtaining a sample of blood from a subject;
applying the sample to a capillary electrophoresis apparatus; and
performing electrophoretic separation and detection of the sample for determining the amount of glycated hemoglobin in the sample, thereby determining whether the subject has diabetes,
wherein the capillary electrophoresis analysis apparatus comprises
an electrophoresis chip comprising
a substrate;
a capillary channel; and
a plurality of liquid reservoirs in communication with each other via the capillary channel;
a voltage application unit comprising an electrode in communication with the capillary channel; and
an absorbance measurement unit, and
wherein the detection is measured by the absorbance measurement unit.

44. The method according to claim 43, wherein the apparatus has a width of about 10 cm to about 100 cm, a depth of about 10 cm to about 100 cm and a height of about 5 cm to about 100 cm.

45. The method according to claim 43, wherein the capillary channel is formed on the surface of the substrate or is a tube embedded in the substrate.

46. The method according to claim 43, wherein an inner wall surface of the capillary channel is coated with a cationic layer, an anionic layer or a neutral layer.

47. The method according to claim 43, wherein the plurality of liquid reservoirs are depressions formed on the surface of the substrate.

48. The method according to claim 43, wherein the electrophoresis chip has a length of about 10 mm to about 100 mm, a width of about 10 mm to about 60 mm and a thickness of about 0.3 mm to about 5 mm.

49. The method according to claim 43, wherein the capillary channel contains an electrophoresis running buffer, wherein the electrophoresis running buffer comprises a sulfated polysaccharide.

50. The method according to claim 49, wherein the sulfated polysaccharide is a chondroitin sulfate.

51. The method according to claim 43, wherein the capillary channel has a diameter of about 25 μm to about 100 μm and a length of about 0.5 cm to about 15 cm.

52. The method according to claim 43, wherein the capillary channel contains a cross-sectional shape perpendicular to the channel direction.

53. The method according to claim 52, wherein the cross-sectional shape is circular, rectangular, ellipsoidal or polygonal.

54. The method according to claim 53, wherein when the cross-sectional shape is circular, the diameter thereof is about 25 μm to about 100 μm.

55. The method according to claim 53, wherein when the cross-sectional shape is rectangular, the width thereof is about 25 μm to about 100 μm and the depth thereof is about 25 μm to about 100 μm.

56. The method according to claim 43, wherein the capillary electrophoresis apparatus further comprises a pre-filter component, an air vent structure, a stray light removing unit, a position adjustment unit, a quantitative dispensing unit, a stirring unit, a liquid sending unit or combinations thereof.

57. The method according to claim 49, wherein the electrophoresis running buffer further comprises a chaotropic anion.

58. The method according to claim 43, wherein the electrophoretic separation and detection of the sample occurs in greater than 0 seconds but less than 35 seconds.
